(19) United States
(12) Patent Application Publication
(10) Pub. No.: US 2008/0300797 A1
(43) Pub. Date: Dec. 4, 2008

(54) TWO BIOMARKERS FOR DIAGNOSIS AND MONITORING OF ATHEROSCLEROTIC CARDIOVASCULAR DISEASE

(75) Inventors: Raymond TABIBIAZAR, Menlo Park, CA (US); Evangelos Hytopoulos, San Mateo, CA (US)

Correspondence Address: MORRISON & FOERSTER LLP, 425 MARKET STREET, SAN FRANCISCO, CA, 94105-2482, US

(73) Assignee: Aviir, Inc., Palo Alto, CA (US)

(21) Appl. No.: 11/963,673

(22) Filed: Dec. 21, 2007

Related U.S. Application Data
(60) Provisional application No. 60/876,614, filed on Dec. 22, 2006.

(51) Int. Cl. G01N 33/48

(52) U.S. Cl. 702/19

(57) ABSTRACT

The present invention identifies two circulating proteins that have been newly identified as being differentially expressed in atherosclerosis. Circulating levels of these two proteins, particularly as a panel of proteins, can discriminate patients with acute myocardial infarction from those with stable exertional angina and from those with no history of atherosclerotic cardiovascular disease. Such levels can also predict cardiovascular events, determine the effectiveness of therapy, stage disease, and the like. For example, these markers are useful as surrogate biomarkers of clinical events needed for development of vascular specific pharmaceutical agents.

### CROSS REFERENCE TO RELATED APPLICATION

This application claims the benefit of U.S. Provisional Application No. 60/876,614, filed Dec. 22, 2006, which is hereby incorporated by reference in its entirety.

### BACKGROUND OF THE INVENTION

1. Field of the Invention

This application is directed to the fields of bioinformatics and atherosclerotic disease. In particular this invention relates to methods and compositions for diagnosing and monitoring atherosclerotic disease.

2. Description of the Related Art

Because of our limited ability to provide early and accurate diagnosis followed by aggressive treatment, atherosclerotic cardiovascular disease (ASCVD) remains the primary cause of morbidity and mortality worldwide. Patients with ASCVD represent a heterogeneous group of individuals, with a disease that progresses at different rates and in distinctly different patterns. Despite appropriate evidence-based treatments for patients with ASCVD, recurrence and mortality rates remain high. Also, the full benefits of primary prevention are unrealized due to our inability to accurately identify those patients who would benefit from aggressive risk reduction.

Whereas certain disease markers have been shown to predict outcome or response to therapy at a population level, they are not sufficiently sensitive or specific to provide adequate clinical utility in an individual patient. As a result, the first clinical presentation for more than half of the patients with coronary artery disease is either myocardial infarction or death.

Physical examination and current diagnostic tools cannot accurately determine an individual's risk for suffering a complication of ASCVD. Known risk factors such as hypertension, hyperlipidemia, diabetes, family history, and smoking do not establish the diagnosis of atherosclerosis disease. Diagnostic modalities which rely on anatomical data (such as coronary angiography, coronary calcium score, CT or MRI angiography) lack information on the biological activity of the disease process and can be poor predictors of future cardiac events. Functional assessment of endothelial function can be non-specific and unrelated to the presence of atherosclerotic disease process, although some data has demonstrated the prognostic value of these measurements. Individual biomarkers, such as the lipid and inflammatory markers, have been shown to predict outcome and response to therapy in patients with ASCVD and some are utilized as important risk factors for developing atherosclerotic disease. Nonetheless, up to this point, no single biomarker is sufficiently specific to provide adequate clinical utility for the diagnosis of ASCVD in an individual patient.

### Complex Nature of Atherosclerotic Cardiovascular Disease

In general, atherosclerosis is believed to be a complex disease involving multiple biological pathways. Variations in the natural history of the atherosclerotic disease process, as well as differential response to risk factors and variations in the individual response to therapy, reflect in part differences in genetic background and their intricate interactions with the environmental factors that are responsible for the initiation and modification of the disease. Atherosclerotic disease is also influenced by the complex nature of the cardiovascular system itself where anatomy, function and biology all play important roles in health as well as disease. Given such complexities, it is unlikely that an individual marker or approach will yield sufficient information to capture the true nature of the disease process.

### Single Biomarker Approach

### Inflammation

Inflammation has been implicated in all stages of ASCVD and is considered to be a major part of the pathophysiological basis of atherogenesis, providing a potential marker of the disease process. Elevated circulating inflammatory biomarkers have been shown to stratify cardiovascular risk and assess response to therapy in large epidemiological studies. Currently, while general markers of inflammation are potentially useful in risk stratification, they are not adequate to identify the presence of CAD in an individual, due a lack of specificity for many markers. For similar reasons, the general markers of inflammation such as C-reactive protein (CRP) and erythrocyte sedimentation rate (ESR) have long been abandoned as specific diagnostic markers in other inflammatory diseases such as lupus and rheumatoid arthritis, although they remain important markers for risk stratification and response to therapy in clinical practice.

It is also possible that the heterogeneity of the individual response to environmental risk factors induces a high variability in ASCVD marker concentration. In this context, biological information carried by a single inflammatory protein cannot be sufficient in providing a comprehensive representation of the vascular inflammatory state, and may not be able to accurately identify the presence or extent of the disease.

### Pathophysiological Basis of Atherosclerosis

Atherosclerotic plaque consists of accumulated intracellular and extracellular lipids, smooth muscle cells, connective tissue, and glycosaminoglycans. The earliest detectable lesion of atherosclerosis is the fatty streak, consisting of lipid-laden foam cells, which are macrophages that have migrated as monocytes from the circulation into the subendothelial layer of the intima, which later evolves into the fibrous plaque, consisting of intimal smooth muscle cells surrounded by connective tissue and intracellular and extracellular lipids. As plaques develop, calcium is deposited.

Interrelated hypotheses have been proposed to explain the pathogenesis of atherosclerosis. The lipid hypothesis postulates that an elevation in plasma LDL levels results in penetration of LDL into the arterial wall, leading to lipid accumulation in smooth muscle cells and in macrophages. LDL also augments smooth muscle cell hyperplasia and migration into the subintimal and intimal region in response to growth factors. LDL is modified or oxidized in this environment and is rendered more atherogenic. The modified or oxidized LDL is chemotactic to monocytes, promoting their migration into the intima, their early appearance in the fatty streak, and their transformation and retention in the subintimal compartment as macrophages. Scavenger receptors on the surface of macrophages facilitate the entry of oxidized LDL into these cells, transferring them into lipid-laden macrophages and foam cells. Oxidized LDL is also cytotoxic to endothelial cells and may be responsible for their dysfunction or loss from the more advanced lesion.

The chronic endothelial injury hypothesis postulates that endothelial injury by various mechanisms produces loss of endothelium, adhesion of platelets to subendothelium, aggregation of platelets, chemotaxis of monocytes and T-cell lymphocytes, and release of platelet-derived and monocyte-derived growth factors that induce migration of smooth muscle cells from the media into the intima, where they replicate, synthesize connective tissue and proteoglycans, and form a fibrous plaque. Other cells, e.g. macrophages, endothelial cells, arterial smooth muscle cells, also produce growth factors that can contribute to smooth muscle hyperplasia and extracellular matrix production.

Endothelial dysfunction includes increased endothelial permeability to lipoproteins and other plasma constituents, expression of adhesion molecules and elaboration of growth factors that lead to increased adherence of monocytes, macrophages and T lymphocytes. These cells may migrate through the endothelium and situate themselves within the subendothelial layer. Foam cells also release growth factors and cytokines that promote migration of smooth muscle cells and stimulate neointimal proliferation, continue to accumulate lipid and support endothelial cell dysfunction. Clinical and laboratory studies have shown that inflammation plays a major role in the initiation, progression and destabilization of atheromas.

The “autoimmune” hypothesis postulates that the inflammatory immunological processes characteristic of the very first stages of atherosclerosis are initiated by humoral and cellular immune reactions against an endogenous antigen. Human Hsp60 expression itself is a response to injury initiated by several stress factors known to be risk factors for atherosclerosis, such as hypertension. Oxidized LDL is another candidate for an autoantigen in atherosclerosis. Antibodies to oxLDL have been detected in patients with atherosclerosis, and they have been found in atherosclerotic lesions. T lymphocytes isolated from human atherosclerotic lesions have been shown to respond to oxLDL and to be a major autoantigen in the cellular immune response. A third autoantigen proposed to be associated with atherosclerosis is 2-Glycoprotein I (2GPI), a glycoprotein that acts as an anticoagulant in vitro. 2GPI is found in atherosclerotic plaques, and hyper-immunization with 2GPI or transfer of 2GPI-reactive T cells enhances fatty streak formation in transgenic atherosclerotic-prone mice.

Infections may contribute to the development of atherosclerosis by inducing both inflammation and autoimmunity. A large number of studies have demonstrated a role of infectious agents, both viruses (cytomegalovirus, herpes simplex viruses, enteroviruses, hepatitis A) and bacteria (C. pneumoniae, H. pylori, periodontal pathogens) in atherosclerosis. Recently, a new “pathogen burden” hypothesis has been proposed, suggesting that multiple infectious agents contribute to atherosclerosis, and that the risk of cardiovascular disease posed by infection is related to the number of pathogens to which an individual has been exposed. Of single micro-organisms, C. pneumoniae probably has the strongest association with atherosclerosis.

These hypotheses are closely linked and not mutually exclusive. Modified LDL is cytotoxic to cultured endothelial cells and may induce endothelial injury, attract monocytes and macrophages, and stimulate smooth muscle growth. Modified LDL also inhibits macrophage mobility, so that once macrophages transform into foam cells in the subendothelial space they may become trapped. In addition, regenerating endothelial cells (after injury) are functionally impaired and increase the uptake of LDL from plasma.

Atherosclerosis is characteristically silent until critical stenosis, thrombosis, aneurysm, or embolus supervenes. Initially, symptoms and signs reflect an inability of blood flow to the affected tissue to increase with demand, e.g. angina on exertion, intermittent claudication. Symptoms and signs commonly develop gradually as the atheroma slowly encroaches on the vessel lumen. However, when a major artery is acutely occluded, the symptoms and signs may be dramatic.

As mentioned above, currently, due to lack of appropriate diagnostic strategies, the first clinical presentation of more than half of the patients with coronary artery disease is either myocardial infarction or death. Further progress in prevention and treatment depends on the development of strategies focused on the primary inflammatory process in the vascular wall, which is fundamental in the etiology of atherosclerotic disease. Without good surrogate markers that accurately report the activity and/or extent of vessel wall disease, methods cannot be developed that completely define risk, monitor the effects of risk reduction toward primary disease amelioration, or develop new classes of therapies that target the vessel wall.

One promising approach is the identification of circulating proteins that reflect the degree and character of vascular inflammation as the hallmark of active cardiovascular disease. A number of immune modulatory proteins have been identified to have some value as surrogate markers, but such biomarkers have not been shown to add sufficient information to have clinical utility. This is due to: i) the failure to consider data on multiple markers measured in parallel, ii) the failure to integrate individual marker data with clinical data that modulates the levels of circulating proteins and obscures the informative patterns, iii) inherited genetic variation that contributes to expression levels of the genes encoding the markers and confounds the abundance measurements, and iv) a lack of information regarding specific immune pathways activated in ASCVD that would better inform biomarker choice. Finally, the prior art fails to provide effective diagnostic or predictive methods using measurements of a panel of circulating proteins.

### Unmet Clinical and Scientific Need

As described above, there is an unmet need for use in clinical medicine and biomedical research for improved tools to identify individuals with vascular inflammation and active atherosclerotic cardiovascular disease. At present, although insights into mechanisms and circumstances of atherosclerosis are increasing, our methods for identifying high-risk patients and predicting the efficacy of prevention strategies remain inadequate. New approaches are needed to better diagnose patients with active atherosclerotic cardiovascular disease at risk for near-term cardiovascular complications. Identification of such patients can lead to initiation of much needed therapies that can result in improved clinical outcomes. The present invention addresses these and other shortcomings of the prior art.

### SUMMARY OF THE DISCLOSURE

The disclosure provides methods, compositions and kit for generating a result useful in diagnosing and monitoring atherosclerotic disease using one or more samples obtained from a mammalian subject. A preferred form of such methods includes obtaining a dataset associated the one or more samples. A preferred dataset has protein expression levels for at least three markers, though in other forms there may be at least four markers, at least five markers, at least six markers, at least eight markers, at least ten markers, at least fifteen markers or at least twenty markers. Preferred markers are the proteins RANTES, TIMP 1, MCP-1, MCP-2, MCP-3, MCP-4, eotaxin, IP-10, M-CSF, IL-3, TNFa, Ang-2, IL-5, IL-7, IGF-1, sVCAM, sICAM-1, E-selectin, P-selection, interleukin-6, interleukin-18, creatine kinase, LDL, oxLDL, LDL particle size, Lipoprotein(a), troponin I, troponin T, LPPLA2, CRP, HDL, Triglyceride, insulin, BNP, fractalkine, osteopontin, osteoprotegerin, oncostatin-M, Myeloperoxidase, ADMA, PAI-1 (plasminogen activator inhibitor), SAA (circulating amyloid A), t-PA (tissue-type plasminogen activator), sCD40 ligand, fibrinogen, homocysteine, D-dimer, leukocyte count, heart-type fatty acid binding protein, Lipoprotein (a), MMP1, Plasminogen, folate, vitamin B6, Leptin, soluble thrombomodulin, PAPPA, MMP9, MMP2, VEGF, PIGF, HGF, vWF, and cystatin C. More preferably, the dataset will include protein expression levels of the protein markers RANTES and/or TIMP1. After the dataset has been obtained it is preferably input into an analytical process that uses the quantitative data to generate a result useful in diagnosing and monitoring atherosclerotic disease.

Another preferred set of protein markers is RANTES, TIMP1, MCP-1, MCP-2, MCP-3, MCP-4, eotaxin, IP-10, M-CSF, IL-3, TNFa, Ang-2, IL-5, IL-7, and IGF-1. In certain aspects, the result will be a classification, a continuous variable or a vector. Such classifications may include two or more classes, three or more classes, four or more classes, or five or more classes. An exemplary classification is a pseudo coronary calcium score where the two or more classes are a low coronary calcium score and a high coronary calcium score.

Preferred forms of the analytical process are a linear algorithm, a quadratic algorithm, a polynomial algorithm, a decision tree algorithm, a voting algorithm, a Linear Discriminant Analysis model, a support vector machine classification algorithm, a recursive feature elimination model, a prediction analysis of microarray model, a Logistic Regression model, a CART algorithm, a FlexTree algorithm, a LART algorithm, a random forest algorithm, a MART algorithm, or Machine Learning algorithms. The analytical processes may use a predictive model or may involve comparing the obtained dataset with a reference dataset. In certain aspects, the reference dataset may be data obtained from one or more healthy control subjects or from one or more subjects diagnosed with an atherosclerotic disease. Comparing the reference dataset to the obtained dataset may include obtaining a statistical measure of a similarity of said obtained dataset to said reference dataset, which may be a comparison of at least three parameters of said obtained dataset to corresponding parameters from said reference dataset.

In certain aspects, the classes may be an atherosclerotic cardiovascular disease classification, a healthy classification, a medication exposure classification, a no medication exposure classification, a low coronary calcium score and a high coronary calcium score.

Additional examples of sets of protein markers to select from in the practice of the disclosed methods includes RANTES, TIMP1, MCP-1, IGF-1, TNFa, M-CSF, Ang-2, and MCP-4; RANTES, TIMP1, MCP-1, MCP-2, MCP-3, MCP-4, eotaxin, IP-10, M-CSF, IL-3, TNFa, Ang-2, IL-5, IL-7, and IGF-1; RANTES, TIMP1, MCP-1, IGF-1, TNFa, IL-5; MCP-1, IGF-1, M-CSF, MCP-2; ANG-2, IGF-1, M-CSF, IL-5; MCP-1, IGF-1, TNFa, MCP-2; MCP-4, IGF-1, M-CSF, IL-5; RANTES, TIMP1, MCP-1, MCP-2, MCP-3, MCP-4, eotaxin, IP-10, M-CSF, IL-3, TNFa, Ang-2, IL-5, IL-7, and IGF-1; and MCP1, MCP2, MCP3, MCP4, Eotaxin, IP10, MCSF, IL3, TNFα, ANG2, IL5, IL7, IGF1, IL10, INFγ, VEGF, MIP1a, RANTES, IL6, IL8, ICAM-1, TIMP1, CCL19, TCA4/6kine/CCL21, CSF3, TRANCE, IL2, IL4, IL13, Il1b, CXCL1/GRO1, GROalpha, IL12, and Leptin.

Preferred analytical processes will provide a quality metric of at least 0.7, at least 0.75, at least 0.8, at least 0.85, or at least 0.9, where preferred quality metrics are AUC and accuracy. Additionally, preferred analytical processes will provide at least one of sensitivity or specificity of at least 0.65, at least 0.7, or at least 0.75.

Preferred atherosclerotic cardiovascular disease classifications to be monitored and/or diagnosed are coronary artery disease, myocardial infarction, and angina. The methods disclosed herein may be used, for example, for classification for atherosclerosis diagnosis, atherosclerosis staging, atherosclerosis prognosis, vascular inflammation levels, assessing extent of atherosclerosis progression, monitoring a therapeutic response, predicting a coronary calcium score, or distinguishing stable from unstable manifestations of atherosclerotic disease.

In addition to the other markers disclosed herein, the markers may be selected from one or more clinical indicia, examples of which are age, gender, LDL concentration, HDL concentration, triglyceride concentration, blood pressure, body mass index, CRP concentration, coronary calcium score, waist circumference, tobacco smoking status, previous history of cardiovascular disease, family history of cardiovascular disease, heart rate, fasting insulin concentration, fasting glucose concentration, diabetes status, and use of high blood pressure medication.

This invention provides methods for detection of circulating protein expression for diagnosis, monitoring, and development of therapeutics, with respect to atherosclerotic conditions, including but not limited to conditions that lead to angina, unstable angina, acute coronary syndrome, myocardial infarction, and heart failure. Specifically, circulating proteins are identified and described herein that are differentially expressed in atherosclerotic patients, including but not limited to circulating inflammatory markers. Circulating inflammatory markers identified herein include MCP-1, MCP-2, MCP-3, MCP-4, eotaxin, IP-10, M-CSF, IL-3, TNFa, Ang-2, IL-5, IL-7, and IGF-1.

The detection of circulating levels of proteins identified herein, which are specifically produced in the vascular wall as a result of the atherosclerotic process, can classify patients as belonging to atherosclerotic conditions, including atherosclerotic disease, no disease, myocardial infarction, stable angina, treatment with medication, no treatment, and the like. Such classification can also be used in prediction of cardiovascular events and response to therapeutics; and are useful to predict and assess complications of cardiovascular disease.

In one embodiment of the invention, the expression profile of a panel of proteins is evaluated for conditions indicative of various stages of atherosclerosis and clinical sequelae thereof. Such a panel provides a level of discrimination not found with individual markers. In one embodiment, the expression profile is determined by measurements of protein concentrations or amounts.

Methods of analysis may include, without limitation, utilizing a dataset to generate a predictive model, and inputting test sample data into such a model in order to classify the sample according to an atherosclerotic classification, where the classification is selected from the group consisting of an atherosclerotic disease classification, a healthy classification, a vascular inflammation classification, a medication exposure classification, a no medication exposure classification, and a coronary calcium score classification, and classifying the sample according to the output of the process. In some embodiments, such a predictive model is used in classifying a sample obtained from a mammalian subject by obtaining a dataset associated with a sample, wherein the dataset comprises at least three, or at least four, or at least five protein markers selected from the group consisting of TIMP1, RANTES, MCP1; MCP2; MCP3; MCP4; Eotaxin; IP10; MCSF; IL3; TNFa; ANG2; IL5; IL7; IGF1; IL10; INFEy; VEGF; MIP1a; RANTES; IL6; IL8; ICAM-1; TIMP1; IL2; IL4; IL13; and Il1b. The data optionally includes a profile for clinical indicia; additional protein expression profiles; metabolic measures, genetic information, and the like.

A predictive model of the invention utilizes quantitative data, such as protein expression levels, from one or more sets of markers described herein. In some embodiments a predictive model provides for a level of accuracy in classification; i.e. the model satisfies a desired quality threshold. A quality threshold of interest may provide for an accuracy or AUC of a given threshold, and either or both of these terms (AUC; accuracy) may be referred to herein as a quality metric. A predictive model may provide a quality metric, e.g. accuracy of classification or AUC, of at least about 0.7, at least about 0.8, at least about 0.9, or higher. Within such a model, parameters may be appropriately selected so as to provide for a desired balance of sensitivity and selectivity.

In other embodiments, analysis of circulating proteins is used in a method of screening biologically active agents for efficacy in the treatment of atherosclerosis. In such methods, cells associated with atherosclerosis, e.g. cells of the vessel wall, etc., are contacted in culture or in vivo with a candidate agent, and the effect on expression of one or more of the markers, e.g. a panel of markers, is determined. In another embodiment, analysis of differential expression of the above circulating proteins is used in a method of following therapeutic regimens in patients. In a single time point or a time course, measurements of expression of one or more of the markers, e.g. a panel of markers, is determined when a patient has been exposed to a therapy, which may include a drug, combination of drugs, non-pharmacologic intervention, and the like.

In another method, relative quantitative measures of 3 or more of atherosclerosis associated proteins identified herein are used to diagnose or monitor atherosclerotic disease in an individual. This panel of proteins identified herein can further include other clinical indicia; additional protein expression profiles; metabolic measures, genetic information, and the like.

In another embodiment, the invention includes methods for classifying a sample obtained from a mammalian subject by obtaining a dataset associated with a sample, wherein the dataset comprises protein expression levels for at least three, or at least four, or at least five, or at least six, or at least seven, or at least eight, or at least nine, or more than nine protein markers selected from the group consisting of TIMP1, RANTES, MCP-1, MCP-2, MCP-3, MCP-4, eotaxin, IP-10, M-CSF, IL-3, TNFa, Ang-2, IL-5, IL-7, and IGF-1, inputting the data into an analytical process that uses the data to classify the sample, where the classification is selected from the group consisting of an atherosclerotic disease classification, a healthy classification, a vascular inflammation classification, a medication exposure classification, a no medication exposure classification, and a coronary calcium score classification, and classifying the sample according to the output of the process.

In another embodiment, the invention includes methods for classifying a sample obtained from a mammalian subject by obtaining a dataset associated with a sample, wherein the dataset comprises protein expression levels for at least three, or at least four, or at least five, or at least six, protein markers that each shows a correlation between a circulating protein concentration and an atherosclerotic vascular tissue RNA concentration, inputting the data into an analytical process that uses the data to classify the sample, where the classification is selected from the group consisting of an atherosclerotic disease classification, a healthy classification, a vascular inflammation classification, a medication exposure classification, a no medication exposure classification, and a coronary calcium score classification, and classifying the sample according to the output of the process.

### BRIEF DESCRIPTION OF THE DRAWINGS

FIG. 1 shows term selection for a Logistic regression model using cross-validation. A model including TIMP1, MCP-1 and RANTES satisfies the expected AUC threshold of 0.85.

FIG. 2 shows the term selection for a Linear discriminant analysis model using cross-validation. A model including TIMP1, MCP-1 and RANTES satisfies the expected AUC threshold of 0.85.

FIG. 3 shows the term selection for a Logistic regression model using cross-validation for the classification of subjects with CCS<10 vs. those with CCS>400

FIG. 4 shows the term selection for a Logistic regression model using the AIC criterion for the classification of subjects with CCS<10 vs. those with CCS>400

FIG. 5a shows Marker selection for a Logistic Regression model using Akaike Information Criterion (AIC).

FIG. 5b shows expected AUC value and S.E. for a series of Logistic Regression models involving an increasing number of terms in the order given in the figure (=inverse order of term removal from the complete model by applying the AIC criterion in the marker selection process).

FIG. 6 shows a Logistic regression model including both clinical variables and biological markers.

FIG. 7 shows a Logistic regression model including alternate clinical variables and biological markers. A model including “Beta Blockers” (DC512) and “Statins” (DC3005) and MCP-4 produces an expected value of AUC in excess of 0.85.

FIG. 8 shows boxplots of value distribution of the first discriminant variate for the three groups: “Untreated,” “ACE or Statins,” and “ACE and Statins.”

FIG. 9 shows the general method applied using 10-fold cross-validation to select an optimum set of markers with an optimum analytical process.

FIG. 10 shows a demonstration of the 10-fold cross-validation approach to select an optimum set of markers using accuracy as a selection criterion.

### DETAILED DESCRIPTION OF THE INVENTION

### Overview

The methods of this invention are useful for diagnosing and monitoring atherosclerotic disease. Atherosclerotic disease is also known as atherosclerosis, arteriosclerosis, atheromatous vascular disease, arterial occlusive disease, or cardiovascular disease, and is characterized by plaque accumulation on vessel walls and vascular inflammation. Vascular inflammation is hallmark of active atherosclerotic disease, unstable plaque, or vulnerable plaque. The plaque consists of accumulated intracellular and extracellular lipids, smooth muscle cells, connective tissue, inflammatory cells, and glycosaminoglycans. Certain plaques also contain calcium. Unstable or active or vulnerable plaques are enriched with inflammatory cells.

By way of example, the present invention includes methods for generating a result useful in diagnosing and monitoring atherosclerotic disease by obtaining a dataset associated with a sample, where the dataset at least includes quantitative data (typically protein expression levels) about protein markers which Applicants have identified as predictive of atherosclerotic disease, and inputting the dataset into an analytic process that uses the dataset to generate a result useful in diagnosing and monitoring atherosclerotic disease. In certain embodiments, the dataset also includes quantitative data about other protein markers previously identified by others as being predictive of atherosclerotic disease and clinical indicia. This quantitative data about other protein markers may be DNA, RNA, or protein expression levels.

The present invention identifies expression profiles of biomarkers of inflammation that can be used for diagnosis and classification of atherosclerotic cardiovascular disease. The protein markers used in the present invention are those identified using a learning algorithm as being capable of distinguishing between different atherosclerotic classifications, e.g., diagnosis, staging, prognosis, monitoring, therapeutic response, prediction of pseudo-coronary calcium score. Other data useful for making atherosclerotic classifications, such as other protein markers previously identified as being predictive of cardiovascular disease and various clinical indicia, may also be a part of the dataset use to generate a result useful for atherosclerotic classification.

Datasets containing quantitative data, typically protein expression levels, for the various protein markers used in the present invention, and quantitative data for other dataset components (e.g., DNA, RNA, and protein expression levels for markers previously identified as useful by others, measures of clinical indicia) can be inputted into an analytical process and used to generate a result. The analytic process may be any type of learning algorithm with defined parameters, or in other words, a predictive model. Predictive models can be developed for a variety of atherosclerotic classifications by applying learning algorithms to the appropriate type of reference or control data. The result of the analytical process/predictive model can be used by an appropriate individual to take the appropriate course of action. For example, if the classification is “healthy” or “atherosclerotic cardiovascular disease”, then a result can be used to determine the appropriate clinical course of treatment for an individual.

The present invention is also useful for diagnosing and monitoring complications of cardiovascular disease, including myocardial infarction, acute coronary syndrome, stroke, heart failure, and angina. An example of a common complication is myocardial infarction, which refers to ischemic myocardial necrosis usually resulting from abrupt reduction in coronary blood flow to a segment of myocardium. In the great majority of patients with acute MI, an acute thrombus, often associated with plaque rupture, occludes the artery that supplies the damaged area. Plaque rupture occurs generally in arteries previously partially obstructed by an atherosclerotic plaque enriched in inflammatory cells. Altered platelet function induced by endothelial dysfunction and vascular inflammation in the atherosclerotic plaque presumably contributes to thrombogenesis. Myocardial infarction can be classified into ST-elevation and non-ST elevation MI (also referred to as unstable angina). In both forms of myocardial infarction, there is myocardial necrosis. In ST-elevation myocardial infraction there is transmural myocardial injury which leads to ST-elevations on electrocardiogram. In non-ST elevation myocardial infarction, the injury is sub-endocardial and is not associated with ST segment elevation on electrocardiogram. Another example of a common atherosclerotic complication is angina, a condition with symptoms of chest pain or discomfort resulting from inadequate blood flow to the heart.

### DEFINITIONS

Terms used in the claims and specification are defined as set forth below unless otherwise specified.

The term “monitoring” as used herein refers to the use of results generated from datasets to provide useful information about an individual or an individual's health or disease status. “Monitoring” can include, for example, determination of prognosis, risk-stratification, selection of drug therapy, assessment of ongoing drug therapy, determination of effectiveness of treatment, prediction of outcomes, determination of response to therapy, diagnosis of a disease or disease complication, following of progression of a disease or providing any information relating to a patient's health status over time, selecting patients most likely to benefit from experimental therapies with known molecular mechanisms of action, selecting patients most likely to benefit from approved drugs with known molecular mechanisms where that mechanism may be important in a small subset of a disease for which the medication may not have a label, screening a patient population to help decide on a more invasive/expensive test, for example, a cascade of tests from a non-invasive blood test to a more invasive option such as biopsy, or testing to assess side effects of drugs used to treat another indication. In particular, the term “monitoring” can refer to atherosclerosis staging, atherosclerosis prognosis, vascular inflammation levels, assessing extent of atherosclerosis progression, monitoring a therapeutic response, predicting a coronary calcium score, or distinguishing stable from unstable manifestations of atherosclerotic disease.

The term “quantitative data” as used herein refers to data associated with any dataset components (e.g., protein markers, clinical indicia, metabolic measures, or genetic assays) that can be assigned a numerical value. Quantitative data can be a measure of the DNA, RNA, or protein level of a marker and expressed in units of measurement such as molar concentration, concentration by weight, etc. For example, if the marker is a protein, quantitative data for that marker can be protein expression levels measured using methods known to those skill in the art and expressed in mM or mg/dL concentration units.

The term “ameliorating” refers to any therapeutically beneficial result in the treatment of a disease state, e.g., an atherosclerotic disease state, including prophylaxis, lessening in the severity or progression, remission, or cure thereof.

The term “mammal” as used herein includes both humans and non-humans and include but is not limited to humans, non-human primates, canines, felines, murines, bovines, equines, and porcines.

The term “pseudo coronary calcium score” as used herein refers to a coronary calcium score generated using the methods as disclosed herein rather than through measurement by an imaging modality. One of skill in the art would recognize that a pseudo coronary calcium score may be used interchangeably with a coronary calcium score generated through measurement by an imaging modality.

The term percent “identity” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel, FM, et al., Current Protocols in Molecular Biology, 4, John Wiley & Sons, Inc., Brooklyn, New York, A.1E. 1-A.1F.11, 1996-2004).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (www.ncbi.nlm.nih.gov/).

The term “sufficient amount” means an amount sufficient to produce a desired effect, e.g., an amount sufficient to alter a protein expression profile.

The term “therapeutically effective amount” is an amount that is effective to ameliorate a symptom of a disease. A therapeutically effective amount can be a “prophylactically effective amount” as prophylaxis can be considered therapy.

Abbreviations used in this application include the following:

TP=true positive

TN=true negative

FP=false positive

FN=false negative

N=total number of negative samples

P=total number of positive samples

A=total number of samples

Accuracy=(TP+TN)/A

Mean CV error=Mean Misclassification error=1−Mean Accuracy

Sensitivity=TP/P=TP/(TP+FN)

Specificity=TN/N=TN/(TN+FP)

CAD=coronary artery disease; MIP1a=MIP1alpha; LDA=Linear Discriminant

Analysis, MI=myocardial infarction; ASCVD=atherosclerotic cardiovascular disease.

It must be noted that, as used in the specification and the appended claims, the singular forms “a,” “an,” and “the” include plural referents unless the context clearly dictates otherwise.

### General Techniques

The practice of the present invention will employ, unless otherwise indicated, conventional techniques of molecular biology (including recombinant techniques), microbiology, cell biology, and biochemistry, which are within the skill of the art. Such techniques are explained fully in the literature, such as: Molecular Cloning: A Laboratory Manual, vol. 1-3, third edition (Sambrook et al., 2001); Oligonucleotide Synthesis (M. J. Gait, ed., 1984); Methods in Enzymology (Academic Press, Inc.); Current Protocols in Molecular Biology (F. M. Ausubel et al., eds., 1987); PCR Cloning Protocols, (Yuan and Janes, eds., 2002, Humana Press).

### Protein Markers Useful for Various Applications

Protein markers useful for making atherosclerotic classifications, e.g., diagnosis, staging, prognosis, monitoring, therapeutic response, prediction of pseudo-coronary calcium score, were identified using a learning algorithm.

Preferred markers are the proteins RANTES, TIMP1, MCP-1, MCP-2, MCP-3, MCP-4, eotaxin, IP-10, M-CSF, IL-3, TNFa, Ang-2, IL-5, IL-7, IGF-1, sVCAM, sICAM-1, E-selectin, P-selection, interleukin-6, interleukin-18, creatine kinase, LDL, oxLDL, LDL particle size, Lipoprotein(a), troponin I, troponin T, LPPLA2, CRP, HDL, Triglyceride, insulin, BNP, fractalkine, osteopontin, osteoprotegerin, oncostatin-M, Myeloperoxidase, ADMA, PAI-1 (plasminogen activator inhibitor), SAA (circulating amyloid A), t-PA (tissue-type plasminogen activator), sCD40 ligand, fibrinogen, homocysteine, D-dimer, leukocyte count, heart-type fatty acid binding protein, Lipoprotein (a), MMP1, Plasminogen, folate, vitamin B6, Leptin, soluble thrombomodulin, PAPPA, MMP9, MMP2, VEGF, PIGF, HGF, vWF, and cystatin C. More preferably, the dataset will include protein expression levels of the protein markers RANTES and/or TIMP1.

Another preferred set of protein markers is RANTES, TIMP1, MCP-1, MCP-2, MCP-3, MCP-4, eotaxin, IP-10, M-CSF, IL-3, TNFa, Ang-2, IL-5, IL-7, and IGF-1.

Additional examples of sets of protein markers to select from in the practice of the disclosed methods includes RANTES, TIMP1, MCP-1, IGF-1, TNFa, M-CSF, Ang-2, and MCP-4; RANTES, TIMP1, MCP-1, MCP-2, MCP-3, MCP-4, eotaxin, IP-10, M-CSF, IL-3, TNFa, Ang-2, IL-5, IL-7, and IGF-1; RANTES, TIMP1, MCP-1, IGF-1, TNFa, IL-5; MCP-1, IGF-1, M-CSF, MCP-2; ANG-2, IGF-1, M-CSF, IL-5; MCP-1, IGF-1, TNFa, MCP-2; MCP-4, IGF-1, M-CSF, IL-5; RANTES, TIMP1, MCP-1, MCP-2, MCP-3, MCP-4, eotaxin, IP-10, M-CSF, IL-3, TNFa, Ang-2, IL-5, IL-7, and IGF-1; and MCP1, MCP2, MCP3, MCP4, Eotaxin, IP10, MCSF, IL3, TNFα, ANG2, IL5, IL7, IGF1, IL10, INF₇, VEGF, MIP1a, RANTES, IL6, IL8, ICAM-1, TIMP1, CCL19, TCA4/6kine/CCL21, CSF3, TRANCE, IL2, IL4, IL13, II1b, CXCL1/GRO1, GROalpha, IL12, and Leptin.

In addition to the other markers disclosed herein, the markers may be selected from one or more clinical indicia, examples of which are age, gender, LDL concentration, HDL concentration, triglyceride concentration, blood pressure, body mass index, CRP concentration, coronary calcium score, waist circumference, tobacco smoking status, previous history of cardiovascular disease, family history of cardiovascular disease, heart rate, fasting insulin concentration, fasting glucose concentration, diabetes status, and use of high blood pressure medication. Further markers are disclosed in U.S. Ser. application Ser. No. 11/473,826 which is hereby incorporated by reference in its entirety.

Additional information regarding preferred markers is provided in Tables 1A and 1B, which contain information taken from Genbank.

TABLE 1A

| | | | | Human | | |
|---|---|---|---|---|---|---|
| | | | | polynucleotide | Human | Human |
| | | | Locus | accession | polynucleotide | protein |
| Protein | Common Alias | Other names | Link | (refseq) | accession (related) | accession |
| CCL2 | \|\|CCL2\|\|SCYA2\|\|MCP1\|\|MONOCYTE | Chemokine (C-C | 6347 | NM_002982 | AC005549, | NP_002973, |
| | CHEMOTACTIC | motif) ligand 2 | | | AF519531, | P13500, |
| | PROTEIN 1\|\|SMALL | | | | AY357296, D26087, | Q6UZ82 |
| | INDUCIBLE CYTOKINE | | | | M28225, M31626, | |
| | A2\|\|chemokine (C-C motif) | | | | M37719, X60001, | |
| | ligand 2\|\|MONOCYTE | | | | Y18933, AV733621, | |
| | CHEMOTACTIC AND | | | | BC009716, | |
| | ACTIVATING | | | | BG530064, | |
| | FACTOR\|\|CHEMOKINE, | | | | BT007329, M24545, | |
| | CC MOTIF, LIGAND | | | | M26683, M28226, | |
| | 2\|\|MCAF CORONARY | | | | S69738, S71513, | |
| | ARTERY DISEASE, | | | | X14768, BU570769, | |
| | MODIFIER | | | | | |
| | OF\|\|CORONARY | | | | | |
| | ARTERY DISEASE, | | | | | |
| | DEVELOPMENT OF, IN | | | | | |
| | HIV\|\| | | | | | |
| CCL8 | \|\|CCL8\|\|MCP2\|\|SCYA8\|\|MONOCYTE | Chemokine (C-C | 6355 | NM_005623 | AC011193, X99886, | NP_005614, |
| | CHEMOTACTIC | motif) ligand 8 | | | Y18047, Y16645, | P80075 |
| | PROTEIN 2\|\|chemokine (C- | | | | Y10802 | |
| | C motif) ligand | | | | | |
| | 8\|\|CHEMOKINE, CC | | | | | |
| | MOTIF, LIGAND | | | | | |
| | 8\|\|SMALL INDUCIBLE | | | | | |
| | CYTOKINE SUBFAMILY | | | | | |
| | A, MEMBER 8\|\| | | | | | |
| CCL7 | \|\|SCYA7\|\|CCL7\|\|MCP3\|\|MONOCYTE | Chemokine (C-C | 6354 | NM_006273 | AC005549, X72309, | NP_006264, |
| | CHEMOTACTIC | motif) ligand 7 | | | CA306760, | P80098, |
| | PROTEIN 3\|\|SMALL | | | | AF043338, | Q569J6, |
| | INDUCIBLE CYTOKINE | | | | BC070240, | Q7Z7Q8 |
| | A7\|\|chemokine (C-C motif) | | | | BC09235, | |
| | ligand 7\|\|CHEMOKINE, | | | | BC112258, | |
| | CC MOTIF, LIGAND 7\|\| | | | | BC112260, X71087 | |
| CCL13 | \|\|NCC1\|\|SCYA13\|\|MCP4\|\|CCL13 | Chemokine (C-C | 6357 | NM_005408 | AC002482, | NP_005399 |
| | \|\|NEW CC | motif) ligand 13 | | | AC011193, | |
| | CHEMOKINE | | | | AJ000979, | |
| | 1\|\|MONOCYTE | | | | AJ001634, | |
| | CHEMOTACTIC | | | | BC008621, | |
| | PROTEIN 4\|\|chemokine (C- | | | | BT007385, | |
| | C motif) ligand | | | | CR450337, U46767, | |
| | 13\|\|CHEMOKINE, CC | | | | U59808, X98306, | |
| | MOTIF, LIGAND | | | | Z77650, Z77651, | |
| | 13\|\|SMALL INDUCIBLE | | | | U59808, BM991948 | |
| | CYTOKINE SUBFAMILY | | | | | |
| | A, MEMBER 13\|\| | | | | | |
| CCL11 | \|\|SCYA11\|\|CCL11\|\|EOTAX | Chemokine (C-C | 6356 | NM_002986 | AB063614, | NP_002977, |
| | IN\|\|SMALL INDUCIBLE | motif) ligand 11 | | | AB063616, | P51671, |
| | CYTOKINE | | | | AC005549, U34780, | Q6I9T4 |
| | A11\|\|CHEMOKINE, CC | | | | U46572, Z92709, | |
| | MOTIF, LIGAND | | | | BC017850, | |
| | 11\|\|chemokine (C-C motif) | | | | BF197516, | |
| | ligand 11\|\|SMALL | | | | CR457421, D49372, | |
| | INDUCIBLE CYTOKINE | | | | U46573, Z69291, | |
| | SUBFAMILY A, | | | | Z75668, Z75669, | |
| | MEMBER 11\|\| | | | | BG485598 | |
| CXCL10 | \|\|INP10\|\|CXCL10\|\|SCYB10\|\| | Chemokine | 3627 | NM_001565 | AC112719, | NP_001556, |
| | IP10\|\|INTERFERON- | (C—X—C motif) | | | BC021117, M27087, | P02778 |
| | GAMMA-INDUCED | ligand 10 | | | M37435, M64592, | |
| | FACTOR\|\|INTERFERON- | | | | M76453, U22386, | |
| | GAMMA-INDUCIBLE | | | | X05825, BC010954, | |
| | PROTEIN 10\|\|MOB1, | | | | X02530 | |
| | MOUSE, HOMOLOG | | | | | |
| | OF\|\|CHEMOKINE, CXC | | | | | |
| | MOTIF, LIGAND | | | | | |
| | 10\|\|chemokine (C—X—C | | | | | |
| | motif) ligand 10\|\|SMALL | | | | | |
| | INDUCIBLE CYTOKINE | | | | | |
| | SUBFAMILY B, | | | | | |
| | MEMBER 10\|\| | | | | | |
| CSF1 | \|\|CSF1\|\|MCSF\|\|MGC31930\|\| | Colony | 1435 | NM_000757, | AL450468, M11038, | NP_00748, |
| | COLONY-STIMULATING | stimulating factor | | NM_172210, | M11295, M11296, | NP757349, |
| | FACTOR 1\|\|COLONY- | 1 (macrophage) | | NM_172211, | X06106, BC021117, | NP757350, |
| | STIMULATING FACTOR, | | | NM172212 | M27087, M37435, | NP757351, |
| | MACROPHAGE- | | | | M64592, M76453, | P09603, |
| | SPECIFIC\|\|macrophage | | | | U22386, X05825, | Q5VVF2, |
| | colony stimulating | | | | BC021117 | Q5VVF3, |
| | factor\|\|Colony stimulating | | | | | Q5VVF4 |
| | factor 1 | | | | | |
| | (macrophage)\|\|colony | | | | | |
| | stimulating factor 1 isoform | | | | | |
| | a precursor\|\|colony | | | | | |
| | stimulating factor 1 isoform | | | | | |
| | c precursor\|\|colony | | | | | |
| | stimulating factor 1 isoform | | | | | |
| | b precursor\|\| | | | | | |
| IL3 | \|\|IL3\|\|MULTI- | Interleukin 3 | 3562 | NM_000588 | AC004511, | NP_000579, |
| | CSF\|\|Interleukin 3 (colony- | (colony- | | | AC034228, | P08700, |
| | stimulating factor, | stimulating | | | AF365976, | Q6GS87, |
| | multiple)\|\| | factor, multiple) | | | BC066272, | Q6NZ78, |
| | | | | | BC066273, | Q6NZ79 |
| | | | | | BC066274, | |
| | | | | | BC066275, | |
| | | | | | BC066276, | |
| | | | | | BC069472, M14743, | |
| | | | | | M17115, M20137 | |
| TNF | \|\|CACHECTIN\|\|TNFA\|\|TNF | Tumor necrosis | 7124 | NM_000594 | AB088112, | NP_000585, |
| | \|\|TNF, MACROPHAGE- | factor (TNF | | | AB202113, | P01375, |
| | DERIVED\|\|TNF, | superfamily, | | | AF129756, | Q5RT83, |
| | MONOCYTE- | member 2) | | | AJ249755, | Q5STB3, |
| | DERIVED\|\|TUMOR | | | | AJ270944, | Q9UBM5 |
| | NECROSIS FACTOR, | | | | AL662801, | |
| | ALPHA\|\|tumor necrosis | | | | AL662847, | |
| | factor (TNF superfamily, | | | | AL929587, | |
| | member 2)\|\| | | | | AY066019, | |
| | | | | | AY214167, | |
| | | | | | AY799806, | |
| | | | | | BA000025, | |
| | | | | | BX248519, M16441, | |
| | | | | | M26331, X02910, | |
| | | | | | Y14768, Z15026, | |
| | | | | | AF043342, | |
| | | | | | AF098751, | |
| | | | | | AJ227911, | |
| | | | | | AJ251878, | |
| | | | | | AJ251879, | |
| | | | | | BC028148, | |
| | | | | | BI908079, M10988, | |
| | | | | | M35592, X01394, | |
| | | | | | AF043342, BC028148, | |
| | | | | | M10988, X01394 | |
| ANGPT2 | \|\|ANG2\|\|angiopoietin- | Angiopoietin 2 | 285 | NM_001147 | AC018398, | NP_001138, |
| | 2B\|\|Tie2- | | | | AY563557, | O15123, |
| | ligand\|\|ANGPT2\|\|AGPT2\|\|angiopoietin- | | | | AB009865, | Q9H4C0, |
| | 2a\|\|Angiopoietin 2\|\| | | | | AF004327, | Q9H4C1, |
| | | | | | AF187858, | Q9HBP3 |
| | | | | | AF218015, | |
| | | | | | AJ289780, | |
| | | | | | AJ289781, | |
| | | | | | AK075219, | |
| | | | | | BC022490, | |
| | | | | | CR620685 | |
| IL5 | \|\|EDF\|\|IL5\|\|EOSINOPHIL | Interleukin 5 | 3567 | NM_000879 | AC116366, | NP_000870, |
| | DIFFERENTIATION | (colony- | | | AF353265, J02971, | P05113 |
| | FACTOR\|\|Interleukin 5 | stimulating | | | J03478, X12706, | |
| | (colony-stimulating factor, | factor, | | | BC066279, | |
| | eosinophil)\|\| | eosinophil) | | | BC066280, | |
| | | | | | BC066281, | |
| | | | | | BC066282, | |
| | | | | | BC069137, X04688, | |
| | | | | | X12705 | |
| IL7 | \|\|IL7\|\|Interleukin 7\|\| | Interleukin 7 | 3574 | NM_000880 | AC083837, M29053, | NP_000871, |
| | | | | | AB102879, | P13232, |
| | | | | | AB102880, | Q5FBX5, |
| | | | | | AB102882, | Q5FBY5, |
| | | | | | AB102883, | Q5FBY6, |
| | | | | | AB102893, | Q5FBY8, |
| | | | | | AU136355, | Q5FBY9 |
| | | | | | BC032487, | |
| | | | | | BC047698, J04156, | |
| IGF1 | \|\|IGF1\|\|IGF I\|\|INSULIN- | Insulin-like | 3479 | NM_000618 | AC010202, | NP_000609, |
| | LIKE GROWTH FACTOR | growth factor 1 | | | AY260957, | P01343, |
| | I\|\|insulin-like growth factor | (somatomedin C) | | | AY790940, M12659, | P05019, |
| | 1 (somatomedin C)\|\| | | | | M14155, M14156, | Q13429, |
| | | | | | S85346, X03420, | Q14620, |
| | | | | | X03421, X03422, | Q59GC5, |
| | | | | | X03563, AB209184, | Q5U743, |
| | | | | | CR541861, M11568, | Q6LD41, |
| | | | | | M27544, M29644, | Q9NP10, |
| | | | | | M37484, U40870, | Q9UC01 |
| | | | | | X00173, X56773, | |
| | | | | | X56774, X57025 | |

TABLE 1B

| | | | | | | |
|---|---|---|---|---|---|---|
| IL10 | \|\|IL10\|\|CSIF\|\|Interleukin | Interleukin 10 | 3586 | NM_000572 | AF295024, | NP_000563, |
| | 10\|\|CYTOKINE SYNTHESIS | | | | AF418271, | P22301, |
| | INHIBITORY FACTOR\|\| | | | | AL513315, | Q6FGS9, |
| | | | | | DQ217938, U16720, | Q6FGW4, |
| | | | | | X78437, AF043333, | Q6LBF4, |
| | | | | | AY029171, | Q71UZ1, |
| | | | | | BC022315, | Q9BXR7 |
| | | | | | BC104252, | |
| | | | | | BC104253, | |
| | | | | | CR541993, | |
| | | | | | CR542028, M57627 | |
| IFNG | \|\|IFNG\|\|IFG\|\|IFI\|\|Interferon, | Interferon, | 3458 | NM_000619 | AC007458, | NP_000610, |
| | gamma\|\|IFN, IMMUNE\|\| | gamma | | | AF375790, J00219, | P01579, |
| | | | | | AF506749, | Q14609, |
| | | | | | AY044154, | Q14610, |
| | | | | | AY255837, | Q14611, |
| | | | | | AY255839, | Q14612, |
| | | | | | BC070256, V00543, | Q14613, |
| | | | | | X01992, X13274, | Q14614, |
| | | | | | X62468, X62469, | Q14615, |
| | | | | | X62470, X62471, | Q53ZV4, |
| | | | | | X62472, X62473, | Q8NHY9, |
| | | | | | X62474, X87308 | Q96LA2 |
| VEGF | \|\|VEGF\|\|Vascular endothelial | Vascular | 7422 | NM_001025366, | AF095785, | NP_003367, |
| | growth factor\|\|VEGFA | endothelial | | NM_001025367, | AF437895, | NP_001020537, |
| | ATHEROSCLEROSIS, | growth factor | | NM_001025368, | AL136131, M63978, | NP_001020538, |
| | SUSCEPTIBILITY TO\|\| | | | NM_001025369, | S85224, AB021221, | NP_001020539, |
| | | | | NM_001025370, | AB209485, | NP_001020540, |
| | | | | NM_001033756, | AF022375, | NP_001020541, |
| | | | | NM_003376 | AF024710, | NP001028928, |
| | | | | | AF062645, | P15692, |
| | | | | | AF091352, | Q59FH5, |
| | | | | | AF214570, | Q6WZM0, |
| | | | | | AF323587, | Q71S09, |
| | | | | | AF430806, | Q96FD9, |
| | | | | | AF486837, | Q9UNS8 |
| | | | | | AJ010438, | |
| | | | | | AK056914, | |
| | | | | | AK125666, | |
| | | | | | AY047581, | |
| | | | | | AY263145, | |
| | | | | | AY500353, | |
| | | | | | AY766116, | |
| | | | | | BC011177, | |
| | | | | | BC019867, | |
| | | | | | BC058855, | |
| | | | | | BC065522, | |
| | | | | | BQ880667, | |
| | | | | | BU153227, | |
| | | | | | CN256173, | |
| | | | | | CR614384, | |
| | | | | | CX756573, M27281, | |
| | | | | | M32977, S85192, | |
| | | | | | X62568 | |
| CCL3 | \|\|SCYA3\|\|CCL3\|\|MIP1A\|\|LD78- | Chemokine | 6348 | NM_002983 | AC069363, D90144, | NP_002974, |
| | ALPHA\|\|MACROPHAGE | (C-C motif) | | | M23178, X04018, | P10147, |
| | INFLAMMATORY | ligand 3 | | | AF043339, | Q14745 |
| | PROTEIN 1- | | | | BC071834, D00044, | |
| | ALPHA\|\|SMALL | | | | D63785, M23452, | |
| | INDUCIBLE CYTOKINE | | | | M25315, X03754, | |
| | A3\|\|chemokine (C-C motif) | | | | CR591007 | |
| | ligand 3\|\|CHEMOKINE, CC | | | | | |
| | MOTIF, LIGAND 3\|\| | | | | | |
| CCL5 | \|\|TCP228\|\|SCYA5\|\|CCL5\|\|T | Chemokine | 6352 | NM_002985 | AB023652, | NP_002976, |
| | CELL-SPECIFIC RANTES\|\|T | (C-C motif) | | | AB023653, | P13501, |
| | CELL-SPECIFIC PROTEIN | ligand 5 | | | AB023654, | Q9UBL2 |
| | p228\|\|SMALL INDUCIBLE | | | | AC015849, | |
| | CYTOKINE A5\|\|chemokine | | | | AF088219, | |
| | (C-C motif) ligand | | | | DQ017060, | |
| | 5\|\|CHEMOKINE, CC MOTIF, | | | | AF043341, | |
| | LIGAND 5\|\|REGULATED | | | | AF266753, | |
| | UPON ACTIVATION, | | | | BC008600, | |
| | NORMALLY T- | | | | BG272739, M21121, | |
| | EXPRESSED, AND | | | | BM917378 | |
| | PRESUMABLY | | | | | |
| | SECRETED\|\| | | | | | |
| IL6 | \|\|IL6\|\|IFNB2\|\|HSF\|\|BSF2\|\|INTERFERON, | Interleukin 6 | 3569 | NM_000600 | AC073072, | NP_000591, |
| | BETA- | (interferon, | | | AF372214, | P05231, |
| | 2\|\|HYBRIDOMA GROWTH | beta 2) | | | CH236948, X04402, | Q75MH2, |
| | FACTOR\|\|HEPATOCYTE | | | | Y00081, BC015511, | Q8N6X1 |
| | STIMULATORY | | | | BT019748, | |
| | FACTOR\|\|B-CELL | | | | BT019749, | |
| | DIFFERENTIATION | | | | CR450296, | |
| | FACTOR\|\|B-CELL | | | | CR590965, | |
| | STIMULATORY FACTOR | | | | CR626263, M14584, | |
| | 2\|\|Interleukin 6 (interferon, | | | | M18403, M29150, | |
| | beta 2)\|\|HGF SERUM IL6 | | | | M54894, S56892, | |
| | LEVEL IN INCREASED | | | | X04403, X04430, | |
| | BMI, MODIFIER OF\|\| | | | | X04602, A09363 | |
| IL8 | \|\|SCYB8\|\|GCP1\|\|IL8\|\|CXCL8\|\| | Interleukin 8 | 3576 | NM_000584 | AC112518, | NP_000575, |
| | NAP1\|\|Interleukin | | | | AF385628, D14283, | P10145 |
| | 8\|\|NEUTROPHIL- | | | | M23344, | |
| | ACTIVATING PEPTIDE | | | | M28130AJ227913, | |
| | 1\|\|MONOCYTE-DERIVED | | | | AK131067, | |
| | NEUTROPHIL | | | | BC013615, | |
| | CHEMOTACTIC | | | | BT007067, | |
| | FACTOR\|\|GRANULOCYTE | | | | CR542151, | |
| | CHEMOTACTIC PROTEIN | | | | CR594973, | |
| | 1\|\|CXC CHEMOKINE | | | | CR600500, | |
| | LIGAND 8\|\|SMALL | | | | CR601533, | |
| | INDUCIBLE CYTOKINE | | | | CR601902, | |
| | SUBFAMILY B, MEMBER | | | | CR603686, | |
| | 8\|\| | | | | CR619554, | |
| | | | | | CR623683, | |
| | | | | | CR623827, M17017, | |
| | | | | | M26383, Y00787, | |
| | | | | | Z11686 | |
| ICAM-1 | \|\|ICAM-1\|\|ANTIGEN | Intercellular | 3383 | NM_000201 | AC011511, | NP_000192, |
| | IDENTIFIED BY | adhesion | | | AY225514, M65001, | O00177, |
| | MONOCLONAL | molecule 1 | | | U86814, X57151, | P05362, |
| | ANTIBODY BB2\|\|SURFACE | (CD54), | | | X59286, AF340038, | Q14601, |
| | ANTIGEN OF ACTIVATED | human | | | AF340039, | Q15463, |
| | B CELLS, BB2\|\|intercellular | rhinovirus | | | AK130659, | Q5NKV7, |
| | adhesion molecule 1 (CD54), | receptor | | | BC015969, | Q5NKV8, |
| | human rhinovirus receptor\|\| | | | | BT006854, | Q99930 |
| | | | | | CR617464, J03132, | |
| | | | | | M24283, M55038, | |
| | | | | | M55091, S82847, | |
| | | | | | X06990 | |
| TIMP1 | \|\|TIMP1\|\|HCI\|\|EPA\|\|COLLAGENASE | TIMP | 7076 | NM_003254 | AY932824, D11139, | NP_003245; |
| | INHIBITOR, | metallopeptidase | | | L47361, Z84466, | Q58P21, |
| | HUMAN\|\|TIMP | inhibitor 1 | | | AK074854, | Q5H9A7, |
| | metallopeptidase inhibitor | | | | BC000866, | Q6FGX5, |
| | 1\|\|tissue inhibitor of | | | | BC007097, | Q96QM2, |
| | metalloproteinase 1 (erythroid | | | | BQ181804, | P01033; |
| | potentiating activity, | | | | BU857950, | Q14252; |
| | collagenase inhibitor)\|\| | | | | CR407638, | Q9UCU1 |
| | | | | | CR541982, | |
| | | | | | CR590572, | |
| | | | | | CR593351, | |
| | | | | | CR602090, M12670, | |
| | | | | | M59906, S68252, | |
| | | | | | X02598, X03124, | |
| | | | | | A10416 | |
| CCL19 | \|\|CCL19\|\|ELC\|\|MIP3B\|\|SCYA19 | Chemokine (C- | 6363 | NM_006274 | AJ223410, | NP_006265, |
| | \|\|EBI1-LIGAND | C motif) ligand | | | AL162231, | Q6IBD6, |
| | CHEMOKINE\|\|EXODUS | 19 | | | AB000887, | Q99731 |
| | 3\|\|MACROPHAGE | | | | BC027968, | |
| | INFLAMMATORY | | | | CR456868, | |
| | PROTEIN 3- | | | | CR623730, U77180, | |
| | BETA\|\|CHEMOKINE, CC | | | | U88321, BM720436 | |
| | MOTIF, LIGAND | | | | | |
| | 19\|\|chemokine (C-C motif) | | | | | |
| | ligand 19\|\|SMALL | | | | | |
| | INDUCIBLE CYTOKINE | | | | | |
| | SUBFAMILY A, MEMBER | | | | | |
| | 19\|\| | | | | | |
| CCL21 | \|\|SCYA21\|\|CCL21\|\|SLC\|\|EXODUS | Chemokine (C- | 6366 | NM_002989 | AF030572, | NP_002980, |
| | 2\|\|SECONDARY | C motif) ligand | | | AJ005654, | O00585, |
| | LYMPHOID TISSUE | 21 | | | AL162231, | Q5VZ73, |
| | CHEMOKINE\|\|CHEMOKINE, | | | | AB002409, | Q6ICR7 |
| | CC MOTIF, LIGAND | | | | AF001979, | |
| | 21\|\|chemokine (C-C motif) | | | | AY358887, | |
| | ligand 21\|\|SMALL | | | | BC027918, | |
| | INDUCIBLE CYTOKINE | | | | BI833188, | |
| | SUBFAMILY A, MEMBER | | | | CR450326, | |
| | 21\|\| | | | | CR615435, U88320, | |
| | | | | | BQ712706 | |
| CSF3 | \|\|GCSF\|\|pluripoietin\|\|CSF3\|\|filgrastim | Colony | 1440 | NM_000759, | AC090844, | NP_757374, |
| | \|\|lenograstim\|\|MGC45931\|\| | stimulating | | NM_172219, | AF388025, M13008, | NP000750, |
| | GCSF | factor 3 | | NM_172220 | X03656, BC033245, | NP75373, |
| | \|\|GRANULOCYTE | (granulocyte) | | | CR541891, M17706, | P09919, |
| | COLONY-STIMULATING | | | | X03438, X03655 | Q6FH65, |
| | FACTOR\|\|COLONY- | | | | | Q8N4W3 |
| | STIMULATING FACTOR | | | | | |
| | 3\|\|granulocyte colony | | | | | |
| | stimulating factor\|\|Colony | | | | | |
| | stimulating factor 3 | | | | | |
| | (granulocyte)\|\|colony | | | | | |
| | stimulating factor 3 isoform | | | | | |
| | c\|\|colony stimulating factor 3 | | | | | |
| | isoform a precursor\|\|colony | | | | | |
| | stimulating factor 3 isoform | | | | | |
| | b precursor\|\| | | | | | |
| TNFSF11 | \|\|ODF\|\|OPGL\|\|RANKL\|\|TRANCE | Tumor necrosis | 8600 | NM_003701, | AL139382, | NP_143026, |
| | \|\|TNFSF11\|\|OSTEOPROTEGERIN | factor (ligand) | | NM_033012 | AB037599, | NP_003692, |
| | LIGAND\|\|OSTEOCLAST | superfamily, | | | AB061227, | O14788, |
| | DIFFERENTIATION | member 11 | | | AB064268, | Q54A98, |
| | FACTOR\|\|TNF-RELATED | | | | AB064269, | Q5T9Y4 |
| | ACTIVATION-INDUCED | | | | AB064270, | |
| | CYTOKINE\|\|RECEPTOR | | | | AF013171, | |
| | ACTIVATOR OF NF- | | | | AF019047, | |
| | KAPPA-B LIGAND\|\|Tumor | | | | AF053712, | |
| | necrosis factor (ligand) | | | | BC074823, | |
| | superfamily, member | | | | BC074890, | |
| | 11\|\|TUMOR NECROSIS | | | | | |
| | FACTOR LIGAND | | | | | |
| | SUPERFAMILY, MEMBER | | | | | |
| | 11\|\| | | | | | |
| IL2 | \|\|IL2\|\|TCGF\|\|Interleukin 2\|\|T- | Interleukin 2 | 3558 | NM_000586 | AC022489, | NP_000577, |
| | CELL GROWTH FACTOR\|\| | | | | AF031845, | P60568, |
| | | | | | AF359939, J00264, | Q13169, |
| | | | | | K02056, M13879, | Q16334, |
| | | | | | M22005, M33199, | Q6NZ91, |
| | | | | | X00695, X61155, | Q6NZ93, |
| | | | | | AF228636, | Q6QWN0, |
| | | | | | AF532913, | Q71V48, |
| | | | | | AY283686, | Q7Z7M3, |
| | | | | | AY523040, | Q8NFA4, |
| | | | | | BC066254, | Q9C001 |
| | | | | | BC066255, | |
| | | | | | BC066256, | |
| | | | | | BC066257, | |
| | | | | | BC070338, | |
| | | | | | DQ231169, S77834, | |
| | | | | | S77835, S82692, | |
| | | | | | U25676, V00564, | |
| | | | | | X01586, A14844 | |
| IL4 | \|\|IL4\|\|BSF1\|\|Interleukin 4\|\|B- | Interleukin 4 | 3565 | NM_000589, | AC004039, | NP_758858, |
| | CELL STIMULATORY | | | NM_172348 | AF395008, | P05112, |
| | FACTOR 1\|\| | | | | AF465829, M23442, | Q5FC01, |
| | | | | | X06750, AB102862, | Q6NWP0, |
| | | | | | AF043336, | Q6NZ77, |
| | | | | | BC066277, | Q9UPB9 |
| | | | | | BC066278, | |
| | | | | | BC067514, | |
| | | | | | BC067515, | |
| | | | | | BC070123, M13982, | |
| | | | | | X81851 | |
| IL13 | \|\|IL13\|\|Interleukin 13\|\| | Interleukin 13 | 3596 | NM_002188 | AC004039, | NP_002179, |
| | | | | | AF172149, | P35225, |
| | | | | | AF172150, | Q4VB50, |
| | | | | | AF193838, | Q4VB51, |
| | | | | | AF193839, | Q4VB52, |
| | | | | | AF193840, | Q4VB53 |
| | | | | | AF377331, | |
| | | | | | AF416600, | |
| | | | | | AY008331, | |
| | | | | | AY008332, L13029, | |
| | | | | | L42079, L42080, | |
| | | | | | U10307, U31120, | |
| | | | | | AF043334, | |
| | | | | | BC096138, | |
| | | | | | BC096139, | |
| | | | | | BC096140, | |
| | | | | | BC096141, L06801, | |
| | | | | | X69079 | |
| IL1b | \|\|IL1B\|\|IL1- | Interleukin 1, | 3553 | NM_000576 | AC079753, | NP_000567, |
| | BETA\|\|INTERLEUKIN 1- | beta | | | AY137079, | O43645, |
| | BETA\|\|Interleukin 1, beta\|\| | | | | BN000002, M15840, | P01584, |
| | | | | | X04500, X52430, | Q53X59, |
| | | | | | X52431, AF043335, | Q53XX2 |
| | | | | | BC008678, | |
| | | | | | BT007213, | |
| | | | | | CR407679, K02770, | |
| | | | | | M15330, M54933, | |
| | | | | | X02532, X56087 | |
| CXCL1 | \|\|CXCL1\|\|NAP-3\|\|MGSA- | Chemokine | 2919 | NM_001511 | AC092438, U03018, | NP_001502, |
| | a\|\|SCYB1\|\|GROa\|\|MGSA | (C—X—C motif) | | | X54489, BC011976, | P09341, |
| | alpha\|\|GRO PROTEIN, | ligand 1 | | | BT006880, J03561, | Q6LD34 |
| | ALPHA\|\|MELANOMA | (melanoma | | | X12510, BF032655 | |
| | GROWTH STIMULATORY | growth | | | | |
| | ACTIVITY, | stimulating | | | | |
| | ALPHA\|\|melanoma growth | activity, alpha) | | | | |
| | stimulatory activity | | | | | |
| | alpha\|\|KC CHEMOKINE, | | | | | |
| | MOUSE, HOMOLOG | | | | | |
| | OF\|\|CHEMOKINE, CXC | | | | | |
| | MOTIF, LIGAND 1\|\|GRO1 | | | | | |
| | oncogene (melanoma | | | | | |
| | growth-stimulating | | | | | |
| | activity)\|\|GRO1 oncogene | | | | | |
| | (melanoma growth | | | | | |
| | stimulating activity, | | | | | |
| | alpha)\|\|SMALL | | | | | |
| | INDUCIBLE CYTOKINE | | | | | |
| | SUBFAMILY B, MEMBER | | | | | |
| | 1\|\|chemokine (C—X—C motif) | | | | | |
| | ligand 1 (melanoma growth | | | | | |
| | stimulating activity, alpha)\|\| | | | | | |
| CXCL2 | \|\|MIP2A\|\|GROb\|\|MGSA- | Chemokine | 2920 | NM_002089 | AC093677 | NP_002080, |
| | b\|\|MIP2- | (C—X—C motif) | | | (22698 . . . 24854, | P19875, |
| | ALPHA\|\|SCYB2\|\|CXCL2\|\|MIP- | ligand 2 | | | complement), | Q6FGD6, |
| | 2a\|\|CINC-2a\|\|GRO2 | | | | U03019, AF043340, | Q6LD33 |
| | oncogene\|\|MGSA beta\|\|GRO | | | | BC005276, | |
| | PROTEIN, | | | | BC015753, | |
| | BETA\|\|MACROPHAGE | | | | BC053653, | |
| | INFLAMMATORY | | | | CR542171, | |
| | PROTEIN 2\|\|melanoma | | | | CR617096, M36820, | |
| | growth stimulatory activity | | | | M57731, X53799 | |
| | beta\|\|CHEMOKINE, CXC | | | | | |
| | MOTIF, LIGAND | | | | | |
| | 2\|\|chemokine (C—X—C motif) | | | | | |
| | ligand 2\|\|SMALL | | | | | |
| | INDUCIBLE CYTOKINE | | | | | |
| | SUBFAMILY B, MEMBER | | | | | |
| | 2\|\| | | | | | |
| IL12B | \|\|NKSF2\|\|CLMF2\|\|IL12B\|\|IL12, | Interleukin 12B | 3593 | NM_002187 | AC011418, | NP_002178, |
| | SUBUNIT p40\|\|IL23, | (natural killer | | | AF512686, | P29460, |
| | SUBUNIT p40\|\|NATURAL | cell stimulatory | | | AY008847, | Q8NOX8 |
| | KILLER CELL | factor 2, | | | AY064126, U89323, | |
| | STIMULATORY FACTOR, | cytotoxic | | | AF180563, | |
| | 40-KD | lymphocyte | | | AY046592, | |
| | SUBUNIT\|\|interleukin 12B | maturation | | | AY046593, | |
| | (natural killer cell | factor 2, p40) | | | BC067498, | |
| | stimulatory factor 2, | | | | BC067499, | |
| | cytotoxic lymphocyte | | | | BC067500, | |
| | maturation factor 2, p40)\|\| | | | | BC067501, | |
| | | | | | BC067502, | |
| | | | | | BC074723, M65272, | |
| | | | | | M65290 | |
| LEP | \|\|LEP\|\|Leptin (obesity homolog, | Leptin (obesity | 3952 | NM_000230 | AC018635, AC018662, | NP_000221, |
| | mouse)\|\|LEP OBESE, MOUSE, | homolog, mouse) | | | AY996373, CH236947, | P41159, |
| | HOMOLOG OF\|\| | | | | D63519, D63710, | Q4TVR7, |
| | | | | | DQ054472, U43415, | Q6NT58 |
| | | | | | AF008123, BC060830, | |
| | | | | | BC069323, BC069452, | |
| | | | | | BC069527, D49487, | |
| | | | | | U18915, U43653 | |

In addition to the specific biomarker sequences identified in this application by name, accession number, or sequence, the invention also contemplates use of biomarker variants that are at least 90% or at least 95% or at least 97% identical to the exemplified sequences and that are now known or later discovered and that have utility for the methods of the invention. These variants may represent polymorphisms, splice variants, mutations, and the like.

### Identification of Additional Protein Markers

Additional protein markers useful for making atherosclerotic classifications may be identified using learning algorithms known in the art (described in further detail in the section entitled “Learning Algorithms”) or other methods known in the art for identifying useful markers, such a imaging or differential expression of mRNA expression levels.

For example, in vivo imaging may be utilized to detect the presence of atherosclerosis associated proteins in heart tissue. Such methods may utilize, for example, labeled antibodies or ligands specific for such proteins. In these embodiments, a detectably-labeled moiety, e.g., an antibody, ligand, etc., which is specific for the polypeptide is administered to an individual (e.g., by injection), and labeled cells are located using standard imaging techniques, including, but not limited to, magnetic resonance imaging, computed tomography scanning, and the like. Detection may utilize one or a cocktail of imaging reagents.

Alternatively, an mRNA sample from vessel tissue, preferably from one or more vessels affected by atherosclerosis, can be analyzed for a genetic signature indicating atherosclerosis in order to identify other protein markers useful for atherosclerotic classification.

In a preferred embodiment, additional useful protein markers are identified by determining the biological pathways which known protein markers are a part of and identifying other markers in that pathway.

The provided patterns of circulating protein expression characterize the inflammatory signature in atherosclerosis, and further links specific immune related pathways to diabetes and medication therapy. While current data suggests a significant role for inflammation in atherosclerosis, there remains little direct data linking immune pathways in the vessel wall to critical aspects of the disease, including the mechanisms by which risk factors impact the primary inflammatory process, and how medications that modify risk factors such as hypertension and hyperlipidemia may specifically impact inflammation. The present invention identifies expression profiles of biomarkers of inflammation that can be used for diagnosis and classification of atherosclerotic cardiovascular disease.

Each of the above-described markers can be used in combination with other dataset components known to be useful for diagnosing or monitoring cardiovascular disease.

### Other Components of Dataset

The dataset may further include a variety of quantitative data about other circulating markers, clinical indicia, metabolic measures, and genetic assay known to those of skill in the art as being useful for diagnosing or monitoring atherosclerotic disease.

Other circulating markers of interest have been reviewed previously (E. J. Armstrong et al, Circulation. 2006; 113(9):e382-385; E. J. Armstrong et al. Circulation. (2006) 113(8):e289-292; E. J. Armstrong et al. Circulation. (2006) 113(7):e152-155; E. J. Armstrong et al. Circulation. (2006) 113(6):e72-75; P. M. Ridker et al. Circulation. (2004) 109(25 Suppl 1):IV6-19; A. R. Folsom et al. Arch Intern Med. (2006) 166(13):1368-1373; and R. S. Vasan et al. Circulation. (2006) 113(19):2335-2362) and include sVCAM (A. R. Folsom et al. Arch Intern Med. (2006) 166(13): 1368-1373 and R. S. Vasan et al. Circulation. (2006) 113(19):2335-2362); sICAM-1 (A. R. Folsom et al. Arch Intern Med. (2006) 166(13):1368-1373); E-selectin (A. R. Folsom et al. Arch Intern Med. (2006) 166(13):1368-1373); P-selection; interleukin-6 (E. J. Armstrong et al. Circulation. (2006) 113(6):e72-75, and P. M. Ridker et al. Circulation. (2000) 101(15):1767-1772), interleukin-18; creatine kinase; LDL, oxLDL, LDL particle size, Lipoprotein(a); troponin I (M. S. Sabatine et al. Circulation. (2002) 105(15):1760-1763), troponin T (M. S. Sabatine et al. Circulation. (2002) 105(15):1760-1763); LPPLA2 (A. R. Folsom et al. Arch Intern Med. (2006) 166(13):1368-1373 and R. S. Vasan et al. Circulation. (2006) 113(19):2335-2362); CRP (U.S. Pat. No. 6,040,147), HDL, Triglyceride, insulin, BNP (brain naturetic peptide) (M. S. Sabatine et al. Circulation. (2002) 105(15):1760-1763), fractalkine, osteopontin, osteoprotegerin (E. J. Rhee et al. Clin Sci (Lond). (2004) 108(3):237-243.), oncostatin-M, Myeloperoxidase (M. L. Brennan et al. N Engl J. Med. (2003) 349(17):1595-1604), ADMA, PAI-1 (plasminogen activator inhibitor), SAA (circulating amyloid A) (R. S. Vasan et al. Circulation. (2006) 113(19):2335-2362), t-PA (tissue-type plasminogen activator)(R. S. Vasan et al. Circulation. (2006) 113(19):2335-2362), sCD40 ligand (E. J. Armstrong et al. Circulation. (2006) 113(6):e72-75), fibrinogen (E. Ernst et al. Ann Intern Med. (1993) 118(12):956-963 and W. B. Kannel et al. The Framingham Study. Jama. (1987) 258(9):1183-1186), homocysteine, D-dimer, leukocyte count (G. D. Friedman et al. N Engl J. Med. (1974) 290(23):1275-1278), heart-type fatty acid binding protein (M. O'Donoghue et al. Circulation. Aug. 8, 2006; 114(6):550-557), Lipoprotein (a), MMP1 (A. R. Folsom et al. Arch Intern Med. (2006) 166(13):1368-1373), Plasminogen (A. R. Folsom et al. Arch Intern Med. (2006) 166(13):1368-1373), folate (A. R. Folsom et al. Arch Intern Med. (2006) 166(13):1368-1373), vitamin B6 (A. R. Folsom et al. Arch Intern Med. (2006) 166(13):1368-1373), Leptin (A. R. Folsom et al. Arch Intern Med. (2006) 166(13):1368-1373), soluble thrombomodulin (A. R. Folsom et al. Arch Intern Med. (2006) 166(13):1368-1373), PAPPA (E. J. Armstrong et al. Circulation. (2006) 113(6):e72-75), MMP9 (E. J. Armstrong et al. Circulation. (2006) 113(6):e72-75), MMP2 (E. J. Armstrong et al. Circulation. (2006) 113(6):e72-75), VEGF (E. J. Armstrong et al. Circulation. (2006) 113(6):e72-75), PIGF (E. J. Armstrong et al. Circulation. (2006) 113(6):e72-75), HGF (E. J. Armstrong et al. Circulation. (2006) 113(6):e72-75), vWF (E. J. Armstrong et al. Circulation. (2006) 113(6):e72-75), and cystatin C (R. S. Vasan et al. Circulation. (2006) 113(19):2335-2362).

### Clinical Indicia

Clinical variables will typically be assessed and the resulting data combined in an algorithm with the above described markers. Such clinical markers include, without limitation: gender; age; glucose; insulin; body mass index (BMI); heart rate; waist size; systolic blood pressure; diastolic blood pressure; dyslipidemia; cigarette smoking; and the like.

Additional clinical indicia useful for making atherosclerotic classifications can be identified using learning algorithms known in the art, such as linear discriminant analysis, support vector machine classification, recursive feature elimination, prediction analysis of microarray, logistic regression, CART, FlexTree, LART, random forest, or MART, which are described in further detail in the section entitled “Learning Algorithms”.

### Obtaining Quantitative Data Used to Generate Dataset

Quantitative data is obtained for each component of the dataset and inputted into an analytic process with previously defined parameters (the predictive model) and then used to generate a result.

The data may be obtained via any technique that results in an individual receiving data associated with a sample. For example, an individual may obtain the dataset by generating the dataset himself by methods known to those in the art. Alternatively, the dataset may be obtained by receiving the dataset from another individual or entity. For example, a laboratory professional may generate the dataset while another individual, such as a medical professional, or may input the dataset into an analytic process to generate the result.

One of skill should understand that although reference is made to “a sample” throughout the specification that the quantitative data may be obtained from multiple samples varying in any number of characteristics, such as the method of procurement, time of procurement, tissue origin, etc.

### Quantitative Data Regarding Protein Markers

In methods of generating a result useful for atherosclerotic classification, the expression pattern in blood, serum, etc. of the protein markers provided herein is obtained. The quantitative data associated with the protein markers of interest can be any data that allows generation of a result useful for atherosclerotic classification, including measurement of DNA or RNA levels associated with the markers but is typically protein expression patterns. Protein levels can be measured via any method known to those of skill of art that generates a quantitative measurement either individually or via high-throughput methods as part of an expression profile. For example, a blood derived patient sample, e.g., blood, plasma, serum, etc. may be applied to a specific binding agent or panel of specific binding agents to determine the presence and quantity of the protein markers of interest.

Sample Procurement

Blood samples, or samples derived from blood, e.g. plasma, circulating, etc. are assayed for the presence of expression levels of the protein markers of interest. Typically a blood sample is drawn, and a derivative product, such as plasma or serum, is tested.

Expression Profiling/Patterns of Multiple Markers

The quantitative data associated with the protein markers of interest typically takes the form of an expression pattern. Expression profiles constitute a set of relative or absolute expression values for a number of RNA or protein products corresponding to the plurality of markers evaluated. In various embodiments, expression profiles containing expression patterns at least about two, three, four, or five markers are produced. The expression pattern for each differentially expressed component member of the expression profile may provide a particular specificity and sensitivity with respect to predictive value, e.g., for diagnosis, prognosis, monitoring treatment, etc.

Methods for Obtaining Expression Data

Numerous methods for obtaining expression data are known, and any one or more of these techniques, singly or in combination, are suitable for determining expression patterns and profiles in the context of the present invention.

For example, DNA and RNA expression patterns can be evaluated by northern analysis, PCR, RT-PCR, Taq Man analysis, FRET detection, monitoring one or more molecular beacon, hybridization to an oligonucleotide array, hybridization to a cDNA array, hybridization to a polynucleotide array, hybridization to a liquid microarray, hybridization to a microelectric array, molecular beacons, cDNA sequencing, clone hybridization, cDNA fragment fingerprinting, serial analysis of gene expression (SAGE), subtractive hybridization, differential display and/or differential screening (see, e.g., Lockhart and Winzeler (2000) Nature 405:827-83 6, and references cited therein).

Protein expression patterns can be evaluated by any method known to those of skill in the art which provides a quantitative measure and is suitable for evaluation of multiple markers extracted from samples such as one or more of the following methods: ELISA sandwich assays, mass spectrometric detection, calorimetric assays, binding to a protein array (e.g., antibody array), or fluorescent activated cell sorting (FACS).

One preferred approach involves the use of labeled affinity reagents (e.g., antibodies, small molecules, etc.) that recognize epitopes of one or more protein products in an ELISA, antibody array, or FACS screen. Methods for producing and evaluating antibodies are well known in the art, see, e.g., Coligan, supra; and Harlow and Lane (1989) Antibodies: A Laboratory Manual, Cold Spring Harbor Press, NY (“Harlow and Lane”). Additional details regarding a variety of immunological and immunoassay procedures adaptable to the present embodiment by selection of antibody reagents specific for the products of protein markers described herein can be found in, e.g., Stites and Ten (eds.) (1991) Basic and Clinical Immunology, 7th ed.

High Throughput Expression Assays

A number of suitable high throughput formats exist for evaluating expression patterns. Typically, the term high throughput refers to a format that performs at least about 100 assays, or at least about 500 assays, or at least about 1000 assays, or at least about 5000 assays, or at least about 10,000 assays, or more per day. When enumerating assays, either the number of samples or the number of protein markers assayed can be considered.

Numerous technological platforms for performing high throughput expression analysis are known. Generally, such methods involve a logical or physical array of either the subject samples, or the protein markers, or both. Common array formats include both liquid and solid phase arrays. For example, assays employing liquid phase arrays, e.g., for hybridization of nucleic acids, binding of antibodies or other receptors to ligand, etc., can be performed in multiwell or microtiter plates. Microtiter plates with 96, 384 or 1536 wells are widely available, and even higher numbers of wells, e.g., 3456 and 9600 can be used. In general, the choice of microtiter plates is determined by the methods and equipment, e.g., robotic handling and loading systems, used for sample preparation and analysis. Exemplary systems include, e.g., the ORCA™ system from Beckman-Coulter, Inc. (Fullerton, Calif.) and the Zymate systems from Zymark Corporation (Hopkinton, Mass.).

Alternatively, a variety of solid phase arrays can favorably be employed to determine expression patterns in the context of the invention. Exemplary formats include membrane or filter arrays (e.g., nitrocellulose, nylon), pin arrays, and bead arrays (e.g., in a liquid “slurry”). Typically, probes corresponding to nucleic acid or protein reagents that specifically interact with (e.g., hybridize to or bind to) an expression product corresponding to a member of the candidate library, are immobilized, for example by direct or indirect cross-linking, to the solid support. Essentially any solid support capable of withstanding the reagents and conditions necessary for performing the particular expression assay can be utilized. For example, functionalized glass, silicon, silicon dioxide, modified silicon, any of a variety of polymers, such as (poly)tetrafluoroethylene, (poly)vinylidenedifluoride, polystyrene, polycarbonate, or combinations thereof can all serve as the substrate for a solid phase array.

In one embodiment, the array is a “chip” composed, e.g., of one of the above-specified materials. Polynucleotide probes, e.g., RNA or DNA, such as cDNA, synthetic oligonucleotides, and the like, or binding proteins such as antibodies or antigen-binding fragments or derivatives thereof, that specifically interact with expression products of individual components of the candidate library are affixed to the chip in a logically ordered manner, i.e., in an array. In addition, any molecule with a specific affinity for either the sense or anti-sense sequence of the marker nucleotide sequence (depending on the design of the sample labeling), can be fixed to the array surface without loss of specific affinity for the marker and can be obtained and produced for array production, for example, proteins that specifically recognize the specific nucleic acid sequence of the marker, ribozymes, peptide nucleic acids (PNA), or other chemicals or molecules with specific affinity.

Detailed discussion of methods for linking nucleic acids and proteins to a chip substrate, are found in, e.g., U.S. Pat. No. 5,143,854, “Large Scale Photolithographic Solid Phase Synthesis Of Polypeptides And Receptor Binding Screening Thereof,” U.S. Pat. No. 5,837,832, “Arrays Of Nucleic Acid Probes On Biological Chips,” U.S. Pat. No. 6,087,112, “Arrays With Modified Oligonucleotide And Polynucleotide Compositions,” U.S. Pat. No. 5,215,882, “Method Of Immobilizing Nucleic Acid On A Solid Substrate For Use In Nucleic Acid Hybridization Assays,” U.S. Pat. No. 5,707,807, “Molecular Indexing For Expressed Gene Analysis,” U.S. Pat. No. 5,807,522, “Methods For Fabricating Microarrays Of Biological Samples,” U.S. Pat. No. 5,958,342, “Jet Droplet Device,” U.S. Pat. No. 5,994,076, “Methods Of Assaying Differential Expression,” to Chenchik et al., U.S. Pat. No. 6,004,755, “Quantitative Microarray Hybridization Assays,” U.S. Pat. No. 6,048,695, “Chemically Modified Nucleic Acids And Method For Coupling Nucleic Acids To Solid Support,” U.S. Pat. No. 6,060,240, “Methods For Measuring Relative Amounts Of Nucleic Acids In A Complex Mixture And Retrieval Of Specific Sequences Therefrom,” U.S. Pat. No. 6,090,556, “Method For Quantitatively Determining The Expression Of A Gene,” and U.S. Pat. No. 6,040,138, “Expression Monitoring By Hybridization To High Density Oligonucleotide Arrays,” each of which is hereby incorporated in its entirety.

Microarray expression may be detected by scanning the microarray with a variety of laser or CCD-based scanners, and extracting features with numerous software packages, for example, Imagene (Biodiscovery), Feature Extraction Software (Agilent), Scanalyze (Eisen, M. 1999. SCANALYZE User Manual; Stanford Univ., Stanford, Calif. Ver 2.32.), GenePix (Axon Instruments).

High-throughput protein systems include commercially available systems from Ciphergen Biosystems, Inc. (Fremont, Calif.) such as Protein Chip® arrays and the Schleicher and Schuell protein microspot array (FastQuant Human Chemokine, S&S Bioscences Inc., Keene, N.H., US).

### Quantitative Data Regarding Other Dataset Components

Quantitative data regarding other dataset components, such as clinical indicia, metabolic measures, and genetic assays, can be determined via methods known to those of skill in the art.

Analytic Processes used to Generate Result

The quantitative data thus obtained about the protein markers and other dataset components is then subjected to an analytic process with parameters previously determined using a learning algorithm, i.e., inputted into a predictive model, as in the examples provided herein (Examples 1-5). The parameters of the analytic process may be those disclosed herein or those derived using the guidelines described herein. Learning algorithms such as linear discriminant analysis, recursive feature elimination, a prediction analysis of microarray, logistic regression, CART, FlexTree, LART, random forest, MART, or another machine learning algorithm are applied to the appropriate reference or training data to determine the parameters for analytical processes suitable for a variety of atherosclerotic classifications.

### Analytic Processes

The analytic process used to generate a result may be any type of process capable of providing a result useful for classifying a sample, for example, comparison of the obtained dataset with a reference dataset, a linear algorithm, a quadratic algorithm, a decision tree algorithm, or a voting algorithm.

Various analytic processes for obtaining a result useful for making an atherosclerotic classification are described herein, however, one of skill in the art will readily understand that any suitable type of analytic process is within the scope of this invention.

Prior to input into the analytical process, the data in each dataset is collected by measuring the values for each marker, usually in triplicate or in multiple triplicates. The data may be manipulated, for example, raw data may be transformed using standard curves, and the average of triplicate measurements used to calculate the average and standard deviation for each patient. These values may be transformed before being used in the models, e.g. log-transformed, Box-Cox transformed (see Box and Cox (1964) J. Royal Stat. Soc., Series B, 26:211-246), etc. This data can then be input into the analytical process with defined parameters.

The analytic process may set a threshold for determining the probability that a sample belongs to a given class. The probability preferably is at least 50%, or at least 60% or at least 70% or at least 80% or higher.

In other embodiments, the analytic process determines whether a comparison between an obtained dataset and a reference dataset yields a statistically significant difference. If so, then the sample from which the dataset was obtained is classified as not belonging to the reference dataset class. Conversely, if such a comparison is not statistically significantly different from the reference dataset, then the sample from which the dataset was obtained is classified as belonging to the reference dataset class.

In general, the analytical process will be in the form of a model generated by a statistical analytical method such as those described below. Examples of such analytical processes may include a linear algorithm, a quadratic algorithm, a polynomial algorithm, a decision tree algorithm, a voting algorithm. A linear algorithm may have the form:

$$R = {C}_{0} + {\sum }_{i = 1}^{N} {C}_{i} {x}_{i}$$

Where R is the useful result obtained. C₀ is a constant that may be zero. Cᵢ and xᵢ are the constants and the value of the applicable biomarker or clinical indicia, respectively, and N is the total number of markers.

A quadratic algorithm may have the form:

$$R = {C}_{0} + {\sum }_{i = 1}^{N} {C}_{i} {x}_{i}^{2}$$

Where R is the useful result obtained. C₀ is a constant that may be zero. Cᵢ and xᵢ are the constants and the value of the applicable biomarker or clinical indicia, respectively, and N is the total number of markers.

A polynomial algorithm is a more generalized form a linear or quadratic algorithm that may have the form:

$$R = {C}_{0} + {\sum }_{i = 0}^{N} {C}_{i} {x}_{i}^{{y}_{i}}$$

Where R is the useful result obtained. C₀ is a constant that may be zero. Cᵢ and xᵢ are the constants and the value of the applicable biomarker or clinical indicia, respectively; yᵢ is the power to which xᵢ is raised and N is the total number of markers.

### Use of Reference/Training Datasets to Determine Parameters of Analytical Process

Using any suitable learning algorithm, an appropriate reference or training dataset is used to determine the parameters of the analytical process to be used for classification, i.e., develop a predictive model.

The reference or training dataset to be used will depend on the desired atherosclerotic classification to be determined. The dataset may include data from two, three, four or more classes.

For example, to use a supervised learning algorithm to determine the parameters for an analytic process used to diagnose atherosclerosis, a dataset comprising control and diseased samples is used as a training set. Alternatively, if a supervised learning algorithm is to be used to develop a predictive model for atherosclerotic staging, then the training set may include data for each of the various stages of cardiovascular disease. Further detail regarding the types of the reference/training datasets used to determine certain atherosclerotic classifications is described in further detail in the section entitled “Use of Results Generated by Analytic Process”.

### Statistical Analysis

The following are examples of the types of statistical analysis methods that are available to one of skill in the art to aid in the practice of the disclosed methods. The statistical analysis may be applied for one or both of two tasks. First, these and other statistical methods may be used to identify preferred subsets of the markers and other indicia that will form a preferred dataset. In addition, these and other statistical methods may be used to generate the analytical process that will be used with the dataset to generate the result. Several of statistical methods presented herein or otherwise available in the art will perform both of these tasks and yield a model that is suitable for use as an analytical process for the practice of the methods disclosed herein.

Biomarkers whose corresponding features values (e.g., expression levels) are capable of discriminating between, e.g., healthy and atherosclerotic are identified herein. The identity of these markers and their corresponding features (e.g., expression levels) can be used to develop an analytical process, or plurality of analytical processes, that discriminate between classes of patients. The examples below illustrate how data analysis algorithms can be used to construct a number of such analytical processes. Each of the data analysis algorithms described in the examples use features (e.g., expression values) of a subset of the markers identified herein across a training population that includes healthy and atherosclerotic patients. Specific data analysis algorithms for building an analytical process, or plurality of analytical processes, that discriminate between subjects disclosed herein will be described in the subsections below. Once an analytical process has been built using these exemplary data analysis algorithms or other techniques known in the art, the analytical process can be used to classify a test subject into one of the two or more phenotypic classes (e.g. a healthy or atherosclerotic patient). This is accomplished by applying the analytical process to a marker profile obtained from the test subject. Such analytical processes, therefore, have enormous value as diagnostic indicators.

The disclosed methods provide, in one aspect, for the evaluation of a marker profile from a test subject to marker profiles obtained from a training population. In some embodiments, each marker profile obtained from subjects in the training population, as well as the test subject, comprises a feature for each of a plurality of different markers. In some embodiments, this comparison is accomplished by (i) developing an analytical process using the marker profiles from the training population and (ii) applying the analytical process to the marker profile from the test subject. As such, the analytical process applied in some embodiments of the methods disclosed herein is used to determine whether a test subject has atherosclerosis.

In some embodiments of the methods disclosed herein, when the results of the application of an analytical process indicate that the subject will likely acquire atherosclerosis, the subject is diagnosed as an “atherosclerotic” subject. If the results of an application of an analytical process indicate that the subject will not develop atherosclerosis, the subject is diagnosed as a healthy subject. Thus, in some embodiments, the result in the above-described binary decision situation has four possible outcomes:

(i) truly atherosclerotic, where the analytical process indicates that the subject will develop atherosclerosis and the subject does in fact develop atherosclerosis during the definite time period (true positive, TP);

(ii) falsely atherosclerotic, where the analytical process indicates that the subject will develop atherosclerosis and the subject, in fact, does not develop atherosclerosis during the definite time period (false positive, FP);

(iii) truly healthy, where the analytical process indicates that the subject will not develop atherosclerosis and the subject, in fact, does not develop atherosclerosis during the definite time period (true negative, TN); or

(iv) falsely healthy, where the analytical process indicates that the subject will not develop atherosclerosis and the subject, in fact, does develop atherosclerosis during the definite time period (false negative, FN).

It will be appreciated that other definitions for TP, FP, TN, UN can be made. While all such alternative definitions are within the scope of the disclosed methods, for ease of understanding, the definitions for TP, FP, TN, and FN given by definitions (i) through (iv) above will be used herein, unless otherwise stated.

As will be appreciated by those of skill in the art, a number of quantitative criteria can be used to communicate the performance of the comparisons made between a test marker profile and reference marker profiles (e.g., the application of an analytical process to the marker profile from a test subject). These include positive predicted value (PPV), negative predicted value (NPV), specificity, sensitivity, accuracy, and certainty. In addition, other constructs such a receiver operator curves (ROC) can be used to evaluate analytical process performance. As used herein: PPV=TP/(TP+FP), NPV=TN/(TN+FN), specificity=TN/(TN+FP), sensitivity=TP/(TP+FN), and accuracy=certainty=(TP+TN)/N.

Here, N is the number of samples compared (e.g., the number of test samples for which a determination of atherosclerotic or healthy is sought). For example, consider the case in which there are ten subjects for which this classification is sought. Marker profiles are constructed for each of the ten test subjects. Then, each of the marker profiles is evaluated by applying an analytical process, where the analytical process was developed based upon marker profiles obtained from a training population. In this example, N, from the above equations, is equal to 10. Typically, N is a number of samples, where each sample was collected from a different member of a population. This population can, in fact, be of two different types. In one type, the population comprises subjects whose samples and phenotypic data (e.g., feature values of markers and an indication of whether or not the subject developed atherosclerosis) was used to construct or refine an analytical process. Such a population is referred to herein as a training population. In the other type, the population comprises subjects that were not used to construct the analytical process. Such a population is referred to herein as a validation population. Unless otherwise stated, the population represented by N is either exclusively a training population or exclusively a validation population, as opposed to a mixture of the two population types. It will be appreciated that scores such as accuracy will be higher (closer to unity) when they are based on a training population as opposed to a validation population. Nevertheless, unless otherwise explicitly stated herein, all criteria used to assess the performance of an analytical process (or other forms of evaluation of a biomarker profile from a test subject) including certainty (accuracy) refer to criteria that were measured by applying the analytical process corresponding to the criteria to either a training population or a validation population. Furthermore, the definitions for PPV, NPV, specificity, sensitivity, and accuracy defined above can also be found in Draghici, Data Analysis Tools for DNA Microanalysis, 2003, CRC Press LLC, Boca Raton, Ha., pp. 342-343, which is hereby incorporated herein by reference.

In some embodiments, N is more than one, more than five, more than ten, more than twenty, between ten and 100, more than 100, or less than 1000 subjects. An analytical process (or other forms of comparison) can have at least about 99% certainty, or even more, in some embodiments, against a training population or a validation population. In other embodiments, the certainty is at least about 97%, at least about 95%, at least about 90%, at least about 85%, at least about 80%, at least about 75%, at least about 70%, at least about 65%, or at least about 60% against a training population or a validation population. The useful degree of certainty may vary, depending on the particular method. As used herein, “certainty” means “accuracy.” In one embodiment, the sensitivity and/or specificity is at is at least about 97%, at least about 95%, at least about 90%, at least about 85%, at least about 80%, at least about 75%, or at least about 70% against a training population or a validation population. In some embodiments, such analytical processes are used to predict the development of atherosclerosis with the stated accuracy. In some embodiments, such analytical processes are used to diagnoses atherosclerosis with the stated accuracy. In some embodiments, such analytical processes are used to determine a stage of atherosclerosis with the stated accuracy.

The number of features that may be used by an analytical process to classify a test subject with adequate certainty is two or more. In some embodiments, it is three or more, four or more, ten or more, or between 10 and 200. Depending on the degree of certainty sought, however, the number of features used in an analytical process can be more or less, but in all cases is at least two. In one embodiment, the number of features that may be used by an analytical process to classify a test subject is optimized to allow a classification of a test subject with high certainty.

Relevant data analysis algorithms for developing an analytical process include, but are not limited to, discriminant analysis including linear, logistic, and more flexible discrimination techniques (see, e.g., Gnanadesikan, 1977, Methods for Statistical Data Analysis of Multivariate Observations, New York: Wiley 1977, which is hereby incorporated by reference herein in its entirety); tree-based algorithms such as classification and regression trees (CART) and variants (see, e.g., Breiman, 1984, Classification and Regression Trees, Belmont, Calif.: Wadsworth International Group, which is hereby incorporated by reference herein in its entirety); generalized additive models (see, e.g., Tibshirani, 1990, Generalized Additive Models, London: Chapman and Hall, which is hereby incorporated by reference herein in its entirety); and neural networks (see, e.g., Neal, 1996, Bayesian Learning for Neural Networks, New York: Springer-Verlag; and Insua, 1998, Feedforward neural networks for nonparametric regression In: Practical Nonparametric and Semiparametric Bayesian Statistics, pp. 181-194, New York: Springer, which is hereby incorporated by reference herein in its entirety).

In one embodiment, comparison of a test subject's marker profile to a marker profiles obtained from a training population is performed, and comprises applying an analytical process. The analytical process is constructed using a data analysis algorithm, such as a computer pattern recognition algorithm. Other suitable data analysis algorithms for constructing analytical process include, but are not limited to, logistic regression (see below) or a nonparametric algorithm that detects differences in the distribution of feature values (e.g., a Wilcoxon Signed Rank Test (unadjusted and adjusted)). The analytical process can be based upon two, three, four, five, 10, 20 or more features, corresponding to measured observables from one, two, three, four, five, 10, 20 or more markers. In one embodiment, the analytical process is based on hundreds of features or more. Analytical process may also be built using a classification tree algorithm. For example, each marker profile from a training population can comprise at least three features, where the features are predictors in a classification tree algorithm (see below). The analytical process predicts membership within a population (or class) with an accuracy of at least about at least about 70%, of at least about 75%, of at least about 80%, of at least about 85%, of at least about 90%, of at least about 95%, of at least about 97%, of at least about 98%, of at least about 99%, or about 100%.

Suitable data analysis algorithms are known in the art, some of which are reviewed in Hastie et al., supra. In a specific embodiment, a data analysis algorithm of the invention comprises Classification and Regression Tree (CART), Multiple Additive Regression Tree (MART), Prediction Analysis for Microarrays (PAM) or Random Forest analysis. Such algorithms classify complex spectra from biological materials, such as a blood sample, to distinguish subjects as normal or as possessing biomarker expression levels characteristic of a particular disease state. In other embodiments, a data analysis algorithm of the invention comprises ANOVA and nonparametric equivalents, linear discriminant analysis, logistic regression analysis, nearest neighbor classifier analysis, neural networks, principal component analysis, quadratic discriminant analysis, regression classifiers and support vector machines. While such algorithms may be used to construct an analytical process and/or increase the speed and efficiency of the application of the analytical process and to avoid investigator bias, one of ordinary skill in the art will realize that computer-based algorithms are not required to carry out the methods of the present invention.

Analytical processes can be used to evaluate biomarker profiles, regardless of the method that was used to generate the marker profile. For example, suitable analytical process that can be used to evaluate marker profiles generated using gas chromatography, as discussed in Harper, “Pyrolysis and GC in Polymer Analysis,” Dekker, New York (1985). Further, Wagner et al., 2002, Anal. Chem. 74:1824-1835 disclose an analytical process that improves the ability to classify subjects based on spectra obtained by static time-of-flight secondary ion mass spectrometry (TOF-SIMS). Additionally, Bright et al., 2002, J. Microbiol. Methods 48:127-38, hereby incorporated by reference herein in its entirety, disclose a method of distinguishing between bacterial strains with high certainty (79-89% correct classification rates) by analysis of MALDI-TOF-MS spectra. Dalluge, 2000, Fresenius J. Anal. Chem. 366:701-711, hereby incorporated by reference herein in its entirety, discusses the use of MALDI-TOF-MS and liquid chromatography-electrospray ionization mass spectrometry (LC/ESI-MS) to classify profiles of biomarkers in complex biological samples.

### Artificial Neural Network

In some embodiments, a neural network is used. A neural network can be constructed for a selected set of markers. A neural network is a two-stage regression or classification model. A neural network has a layered structure that includes a layer of input units (and the bias) connected by a layer of weights to a layer of output units. For regression, the layer of output units typically includes just one output unit. However, neural networks can handle multiple quantitative responses in a seamless fashion.

In multilayer neural networks, there are input units (input layer), hidden units (hidden layer), and output units (output layer). There is, furthermore, a single bias unit that is connected to each unit other than the input units. Neural networks are described in Duda et al., 2001, Pattern Classification, Second Edition, John Wiley & Sons, Inc., New York; and Hastie et al., 2001, The Elements of Statistical Learning, Springer-Verlag, New York

The basic approach to the use of neural networks is to start with an untrained network, present a training pattern to the input layer, and to pass signals through the net and determine the output at the output layer. These outputs are then compared to the target values; any difference corresponds to an error. This error or criterion function is some scalar function of the weights and is minimized when the network outputs match the desired outputs. Thus, the weights are adjusted to reduce this measure of error. For regression, this error can be sum-of-squared errors. For classification, this error can be either squared error or cross-entropy (deviation). See, e.g., Hastie et al., 2001, The Elements of Statistical Learning, Springer-Verlag, New York, which is hereby incorporated by reference in its entirety.

The basic approach to the use of neural networks is to start with an untrained network, present a training pattern, e.g., marker profiles from training patients, to the input layer, and to pass signals through the net and determine the output, e.g., the prognosis of the training patients, at the output layer. These outputs are then compared to the target values; any difference corresponds to an error. This error or criterion function is some scalar function of the weights and is minimized when the network outputs match the desired outputs. Thus, the weights are adjusted to reduce this measure of error. For regression, this error can be sum-of-squared errors. For classification, this error can be either squared error or cross-entropy (deviation). See, e.g., Hastie et al., 2001, The Elements of Statistical Learning, Springer-Verlag, New York.

Three commonly used training protocols are stochastic, batch, and on-line. In stochastic training, patterns are chosen randomly from the training set and the network weights are updated for each pattern presentation. Multilayer nonlinear networks trained by gradient descent methods such as stochastic back-propagation perform a maximum-likelihood estimation of the weight values in the model defined by the network topology. In batch training, all patterns are presented to the network before learning takes place. Typically, in batch training, several passes are made through the training data. In online training, each pattern is presented once and only once to the net.

In some embodiments, consideration is given to starting values for weights. If the weights are near zero, then the operative part of the sigmoid commonly used in the hidden layer of a neural network (see, e.g., Hastie et al., 2001, The Elements of Statistical Learning, Springer-Verlag, New York) is roughly linear, and hence the neural network collapses into an approximately linear model. In some embodiments, starting values for weights are chosen to be random values near zero. Hence the model starts out nearly linear, and becomes nonlinear as the weights increase. Individual units localize to directions and introduce nonlinearities where needed. Use of exact zero weights leads to zero derivatives and perfect symmetry, and the algorithm never moves. Alternatively, starting with large weights often leads to poor solutions.

Since the scaling of inputs determines the effective scaling of weights in the bottom layer, it can have a large effect on the quality of the final solution. Thus, in some embodiments, at the outset all expression values are standardized to have mean zero and a standard deviation of one. This ensures all inputs are treated equally in the regularization process, and allows one to choose a meaningful range for the random starting weights. With standardization inputs, it is typical to take random uniform weights over the range [−0.7, +0.7].

A recurrent problem in the use of networks having a hidden layer is the optimal number of hidden units to use in the network. The number of inputs and outputs of a network are determined by the problem to be solved. For the methods disclosed herein, the number of inputs for a given neural network can be the number of markers in the selected set of markers. The number of output for the neural network will typically be just one. However, in some embodiment more than one output is used so that more than just two states can be defined by the network. If too many hidden units are used in a neural network, the network will have too many degrees of freedom and is trained too long, there is a danger that the network will overfit the data. If there are too few hidden units, the training set cannot be learned. Generally speaking, however, it is better to have too many hidden units than too few. With too few hidden units, the model might not have enough flexibility to capture the nonlinearities in the data; with too many hidden units, the extra weight can be shrunk towards zero if appropriate regularization or pruning, as described below, is used. In typical embodiments, the number of hidden units is somewhere in the range of 5 to 100, with the number increasing with the number of inputs and number of training cases.

One general approach to determining the number of hidden units to use is to apply a regularization approach. In the regularization approach, a new criterion function is constructed that depends not only on the classical training error, but also on classifier complexity. Specifically, the new criterion function penalizes highly complex models; searching for the minimum in this criterion is to balance error on the training set with error on the training set plus a regularization term, which expresses constraints or desirable properties of solutions:

J=Jₚₐₜ+λJ_{reg}.

The parameter λ is adjusted to impose the regularization more or less strongly. In other words, larger values for λ will tend to shrink weights towards zero: typically cross-validation with a validation set is used to estimate λ. This validation set can be obtained by setting aside a random subset of the training population. Other forms of penalty can also be used, for example the weight elimination penalty (see, e.g., Hastie et al., 2001, The Elements of Statistical Learning, Springer-Verlag, New York).

Another approach to determine the number of hidden units to use is to eliminate—prune—weights that are least needed. In one approach, the weights with the smallest magnitude are eliminated (set to zero). Such magnitude-based pruning can work, but is nonoptimal; sometimes weights with small magnitudes are important for learning and training data. In some embodiments, rather than using a magnitude-based pruning approach, Wald statistics are computed. The fundamental idea in Wald Statistics is that they can be used to estimate the importance of a hidden unit (weight) in a model. Then, hidden units having the least importance are eliminated (by setting their input and output weights to zero). Two algorithms in this regard are the Optimal Brain Damage (OBD) and the Optimal Brain Surgeon (OBS) algorithms that use second-order approximation to predict how the training error depends upon a weight, and eliminate the weight that leads to the smallest increase in training error.

Optimal Brain Damage and Optimal Brain Surgeon share the same basic approach of training a network to local minimum error at weight w, and then pruning a weight that leads to the smallest increase in the training error. The predicted functional increase in the error for a change in full weight vector δw is:

$$\partial J = {( \frac{\partial J}{\partial w} )}^{t} \cdot \partial w + 1 / 2 \partial {w}^{t} \cdot \frac{{\partial }^{2} J}{\partial {w}^{2}} \cdot \partial w + O ( {‖ \partial w ‖}^{3} )$$

$$where$$

$$\frac{{\partial }^{2} J}{\partial {w}^{2}}$$

is the Hessian matrix. The first term vanishes because we are at a local minimum in error; third and higher order terms are ignored. The general solution for minimizing this function given the constraint of deleting one weight is:

$$\partial w = - \frac{{w}_{q}}{{[ {H}^{- 1} ]}_{qq}} {H}^{- 1} \cdot {u}_{q}$$

$$and$$

$${L}_{q} = 1 / 2 - \frac{{w}_{q}^{2}}{{[ {H}^{- 1} ]}_{qq}}$$

Here, u_{q} is the unit vector along the qth direction in weight space and L_{q} is approximation to the saliency of the weight q—the increase in training error if weight q is pruned and the other weights updated δw. These equations require the inverse of H. One method to calculate this inverse matrix is to start with a small value, H₀⁻¹=α⁻¹I, where α is a small parameter—effectively a weight constant. Next the matrix is updated with each pattern according to

$${H}_{m + 1}^{- 1} = {H}_{m}^{- 1} \frac{{H}_{m}^{- 1} {X}_{m + 1} {X}_{m + 1}^{T} {H}_{m}^{- 1}}{\frac{n}{{a}_{m}} + {X}_{m + 1}^{T} {H}_{m}^{- 1} {X}_{m + 1}}$$

where the subscripts correspond to the pattern being presented and am decreases with m. After the full training set has been presented, the inverse Hessian matrix is given by H⁻¹=Hₙ⁻¹. In algorithmic form, the Optimal Brain Surgeon method is:

$${q}^{*} ← arg {min}_{q} {w}_{q}^{2} / ( {2 [ {H}^{- 1} ]}_{qq} ) ( saliency {L}_{q} )$$

$$w ← w - \frac{{w}_{{q}^{*}}}{[ {H}^{- 1} ] {q}^{*} {q}^{*}} {H}^{- 1} {e}_{{q}^{*}} ( saliency {L}_{q} )$$

The Optimal Brain Damage method is computationally simpler because the calculation of the inverse Hessian matrix in line 3 is particularly simple for a diagonal matrix. The above algorithm terminates when the error is greater than a criterion initialized to be θ. Another approach is to change line 6 to terminate when the change in J(w) due to elimination of a weight is greater than some criterion value.

In some embodiments, a back-propagation neural network (see, for example Abdi, 1994, “A neural network primer”, J. Biol System. 2, 247-283) may be used.

### Support Vector Machines

In some embodiments of the present invention, support vector machines (SVMs) are used to classify subjects using feature values of the markers described herein. SVMs are a relatively new type of learning algorithm, which are generally described, for example, in Cristianini and Shawe-Taylor, 2000, An Introduction to Support Vector Machines, Cambridge University Press, Cambridge; Boser et al., 1992, “A training algorithm for optimal margin classifiers,” in Proceedings of the 5th Annual ACM Workshop on Computational Learning Theory, ACM Press, Pittsburgh, Pa., pp. 142-152; Vapnik, 1998, Statistical Learning Theory, Wiley, New York; Mount, 2001, Bioinformatics: sequence and genome analysis, Cold Spring Harbor Laboratory Press, Cold Spring Harbor, N.Y., Duda, Pattern Classification, Second Edition, 2001, John Wiley & Sons, Inc.; and Hastie, 2001, The Elements of Statistical Learning, Springer, New York; and Furey et al., 2000, Bioinformatics 16, 906-914, each of which is hereby incorporated by reference in its entirety. When used for classification, SVMs separate a given set of binary labeled data training data with a hyper-plane that is maximally distance from them. For cases in which no linear separation is possible, SVMs can work in combination with the technique of ‘kernels’, which automatically realizes a non-linear mapping to a feature space. The hyper-plane found by the SVM in feature space corresponds to a non-linear decision boundary in the input space.

In one approach, when a SVM is used, the feature data is standardized to have mean zero and unit variance and the members of a training population are randomly divided into a training set and a test set. For example, in one embodiment, two thirds of the members of the training population are placed in the training set and one third of the members of the training population are placed in the test set. The expression values for a combination of markers described herein is used to train the SVM. Then the ability for the trained SVM to correctly classify members in the test set is determined. In some embodiments, this computation is performed several times for a given combination of markers. In each iteration of the computation, the members of the training population are randomly assigned to the training set and the test set. Then, the quality of the combination of biomarkers is taken as the average of each such iteration of the SVM computation.

### Predictive Analysis of Microarrays (PAM)

One approach to developing an analytical process using expression levels of markers disclosed herein is the nearest centroid classifier. Such a technique computes, for each class (e.g., healthy and atherosclerotic), a centroid given by the average expression levels of the markers in the class, and then assigns new samples to the class whose centroid is nearest. This approach is similar to k-means clustering except clusters are replaced by known classes. This algorithm can be sensitive to noise when a large number of markers are used. One enhancement to the technique uses shrinkage: for each marker, differences between class centroids are set to zero if they are deemed likely to be due to chance. This approach is implemented in the Prediction Analysis of Microarray, or PAM. See, for example, Tibshirani et al., 2002, Proceedings of the National Academy of Science USA 99; 6567-6572, which is hereby incorporated by reference in its entirety. Shrinkage is controlled by a threshold below which differences are considered noise. Markers that show no difference above the noise level are removed. A threshold can be chosen by cross-validation. As the threshold is decreased, more markers are included and estimated classification errors decrease, until they reach a bottom and start climbing again as a result of noise markers—a phenomenon known as overfitting.

### Multiple Additive Regression Trees

Multiple additive regression trees (MART) represents another way to construct an analytical process that can be used in the methods disclosed herein. A generic algorithm for MART is:

1. Initialize

$${F}_{0} ( x ) = arg min y {\sum }_{i = 1}^{N} L ( {y}_{i} , y )$$

2. For m=1 to M:

(a) For I=1, 2, . . . , N compute

$${r}_{im} = - {\vert \frac{\partial L ( {y}_{i} , f ( {x}_{i} ) )}{\partial f ( {x}_{i} )} \vert}_{f = {f}_{m} - 1}$$

(b) Fit a regression tree to the targets rim giving terminal regions Rjm, j=1, 2, . . . , Jm.

(c) For j=1, 2, . . . , Jm compute

$${γ}_{jm} = arg min γ {\sum }_{{x}_{i} ∉ {R}_{jm}} L ( {y}_{i} , {f}_{m - 1} ( {x}_{i} ) + γ )$$

$$( d ) Update fm ( x ) = fm - I ( x ) + {\sum }_{j = 1}^{Jm} {γ}_{jm} I ( x ∈ {R}_{jm} )$$

3. Output f̂(x)=f_{M}(x).

Specific algorithms are obtained by inserting different loss criteria L(y,f(x)). The first line of the algorithm initializes to the optimal constant model, which is just a single terminal node tree. The components of the negative gradient computed in line 2(a) are referred to as generalized pseudo residuals, r. Gradients for commonly used loss functions are summarized in Table 10.2, of Hastie et al., 2001, The Elements of Statistical Learning, Springer-Verlag, New York, p. 321, which is hereby incorporated by reference. The algorithm for classification is similar and is described in Hastie et al., Chapter 10, which is hereby incorporated by reference in its entirety. Tuning parameters associated with the MART procedure are the number of iterations M and the sizes of each of the constituent trees Jₘ, m=1, 2, . . . , M.

### Analytical Processes Derived by Regression

In some embodiments, an analytical process used to classify subjects is built using regression. In such embodiments, the analytical process can be characterized as a regression classifier, preferably a logistic regression classifier. Such a regression classifier includes a coefficient for each of the markers (e.g., the expression level for each such marker) used to construct the classifier. In such embodiments, the coefficients for the regression classifier are computed using, for example, a maximum likelihood approach. In such a computation, the features for the biomarkers (e.g., RT-PCR, microarray data) is used. In particular embodiments, molecular marker data from only two trait subgroups is used (e.g., healthy patients and atherosclerotic patients) and the dependent variable is absence or presence of a particular trait in the subjects for which marker data is available.

In another specific embodiment, the training population comprises a plurality of trait subgroups (e.g., three or more trait subgroups, four or more specific trait subgroups, etc.). These multiple trait subgroups can correspond to discrete stages in the phenotypic progression from healthy, to mild atherosclerosis, to medium atherosclerosis, etc. in a training population. In this specific embodiment, a generalization of the logistic regression model that handles multicategory responses can be used to develop a decision that discriminates between the various trait subgroups found in the training population. For example, measured data for selected molecular markers can be applied to any of the multi-category logit models described in Agresti, An Introduction to Categorical Data Analysis, 1996, John Wiley & Sons, Inc., New York, Chapter 8, hereby incorporated by reference in its entirety, in order to develop a classifier capable of discriminating between any of a plurality of trait subgroups represented in a training population.

### Logistic Regression

In some embodiments, the analytical process is based on a regression model, preferably a logistic regression model. Such a regression model includes a coefficient for each of the markers in a selected set of markers disclosed herein. In such embodiments, the coefficients for the regression model are computed using, for example, a maximum likelihood approach. In particular embodiments, molecular marker data from the two groups (e.g., healthy and diseased) is used and the dependent variable is the status of the patient for which marker characteristic data are from.

Some embodiments of the disclosed methods provide generalizations of the logistic regression model that handle multicategory (polychotomous) responses. Such embodiments can be used to discriminate an organism into one or three or more classifications. Such regression models use multicategory logit models that simultaneously refer to all pairs of categories, and describe the odds of response in one category instead of another. Once the model specifies logits for a certain (J-1) pairs of categories, the rest are redundant. See, for example, Agresti, An Introduction to Categorical Data Analysis, John Wiley & Sons, Inc., 1996, New York, Chapter 8, which is hereby incorporated by reference.

### Linear Discriminant Analysis

Linear discriminant analysis (LDA) attempts to classify a subject into one of two categories based on certain object properties. In other words, LDA tests whether object attributes measured in an experiment predict categorization of the objects. LDA typically requires continuous independent variables and a dichotomous categorical dependent variable. For use with the disclosed methods, the expression values for the selected set of markers across a subset of the training population serve as the requisite continuous independent variables. The group classification of each of the members of the training population serves as the dichotomous categorical dependent variable.

LDA seeks the linear combination of variables that maximizes the ratio of between-group variance and within-group variance by using the grouping information. Implicitly, the linear weights used by LDA depend on how the expression of a marker across the training set separates in the two groups (e.g., a group that has atherosclerosis and a group that does not have atherosclerosis) and how this expression correlates with the expression of other markers. In some embodiments, LDA is applied to the data matrix of the N members in the training sample by K genes in a combination of genes described in the present invention. Then, the linear discriminant of each member of the training population is plotted. Ideally, those members of the training population representing a first subgroup (e.g. those subjects that do not have atherosclerosis) will cluster into one range of linear discriminant values (e.g., negative) and those member of the training population representing a second subgroup (e.g. those subjects that have atherosclerosis) will cluster into a second range of linear discriminant values (e.g., positive). The LDA is considered more successful when the separation between the clusters of discriminant values is larger. For more information on linear discriminant analysis, see Duda, Pattern Classification, Second Edition, 2001, John Wiley & Sons, Inc; and Hastie, 2001, The Elements of Statistical Learning, Springer, New York; Venables & Ripley, 1997, Modern Applied Statistics with s-plus, Springer, New York.

### Quadratic Discriminant Analysis

Quadratic discriminant analysis (QDA) takes the same input parameters and returns the same results as LDA. QDA uses quadratic equations, rather than linear equations, to produce results. LDA and QDA are roughly interchangeable (though there are differences related to the number of subjects required), and which to use is a matter of preference and/or availability of software to support the analysis. Logistic regression takes the same input parameters and returns the same results as LDA and QDA.

### Decision Trees

One type of analytical process that can be constructed using the expression level of the markers identified herein is a decision tree. Here, the “data analysis algorithm” is any technique that can build the analytical process, whereas the final “decision tree” is the analytical process. An analytical process is constructed using a training population and specific data analysis algorithms. Decision trees are described generally by Duda, 2001, Pattern Classification, John Wiley & Sons, Inc., New York. pp. 395-396, which is hereby incorporated by reference. Tree-based methods partition the feature space into a set of rectangles, and then fit a model (like a constant) in each one.

The training population data includes the features (e.g., expression values, or some other observable) for the markers across a training set population. One specific algorithm that can be used to construct an analytical process is a classification and regression tree (CART). Other specific decision tree algorithms include, but are not limited to, ID3, C4.5, MART, and Random Forests. CART, ID3, and C4.5 are described in Duda, 2001, Pattern Classification, John Wiley & Sons, Inc., New York. pp. 396-408 and pp. 411-412, which is hereby incorporated by reference. CART, MART, and C4.5 are described in Hastie et al., 2001, The Elements of Statistical Learning, Springer-Verlag, New York, Chapter 9, which is hereby incorporated by reference in its entirety. Random Forests are described in Breiman, 1999, “Random Forests—Random Features,” Technical Report 567, Statistics Department, U.C. Berkeley, September 1999, which is hereby incorporated by reference in its entirety.

In some embodiments of the disclosed methods, decision trees are used to classify patients using expression data for a selected set of markers. Decision tree algorithms belong to the class of supervised learning algorithms. The aim of a decision tree is to induce an analytical process (a tree) from real-world example data. This tree can be used to classify unseen examples which have not been used to derive the decision tree.

A decision tree is derived from training data. An example contains values for the different attributes and what class the example belongs. In one embodiment, the training data is expression data for a combination of markers described herein across the training population.

The following algorithm describes a decision tree derivation:

Tree (Examples,Class,Attributes)

Create a root node

If all Examples have the same Class value, give the root this label

Else if Attributes is empty label the root according to the most common value

Else begin

Calculate the information gain for each attribute

Select the attribute A with highest information gain and make this the root attribute

For each possible value, v, of this attribute

Add a new branch below the root, corresponding to A=v Let Examples(v) be those examples with A=v

If Examples(v) is empty, make the new branch a leaf node labeled with the most common value among Examples

Else let the new branch be the tree created by Tree (Examples(v),Class,Attributes-{A})

end

A more detailed description of the calculation of information gain is shown in the following. If the possible classes vi of the examples have probabilities P(vi) then the information content I of the actual answer is given by:

$$I ( P ( {V}_{1} ) , … , P ( {V}_{n} ) ) = {\sum }_{i = 1}^{n} - P ( {v}_{i} ) {log}_{2} P ( {v}_{i} )$$

The I-value shows how much information is needed in order to be able to describe the outcome of a classification for the specific dataset used. Supposing that the dataset contains p positive (e.g. has atherosclerosis) and n negative (e.g. healthy) examples (e.g. individuals), the information contained in a correct answer is:

$$I ( \frac{p}{p + n} , \frac{n}{p + n} ) = - \frac{p}{p + n} {log}_{2} \frac{p}{p + n} - \frac{n}{p + n} {log}_{2} \frac{n}{p + n}$$

where log₂ is the logarithm using base two. By testing single attributes the amount of information needed to make a correct classification can be reduced. The remainder for a specific attribute A (e.g. a marker) shows how much the information that is needed can be reduced.

$$Remainder ( A ) = {\sum }_{i = 1}^{v} \frac{{p}_{i} + {n}_{i}}{p + n} I ( \frac{{p}_{i}}{{p}_{i} + {n}_{i}} , \frac{{n}_{i}}{{p}_{i} + {n}_{i}} )$$

where “v” is the number of unique attribute values for attribute A in a certain dataset, “i” is a certain attribute value, “pᵢ” is the number of examples for attribute A where the classification is positive (e.g. atherosclerotic), “nᵢ” is the number of examples for attribute A where the classification is negative (e.g. healthy).

The information gain of a specific attribute A is calculated as the difference between the information content for the classes and the remainder of attribute A:

$$Gain ( A ) = I ( \frac{p}{p + n} , \frac{n}{p + n} ) - Remainder ( A )$$

The information gain is used to evaluate how important the different attributes are for the classification (how well they split up the examples), and the attribute with the highest information.

In general there are a number of different decision tree algorithms, many of which are described in Duda, Pattern Classification, Second Edition, 2001, John Wiley & Sons, Inc. Decision tree algorithms often require consideration of feature processing, impurity measure, stopping criterion, and pruning. Specific decision tree algorithms include, cut are not limited to classification and regression trees (CART), multivariate decision trees, ID3, and C4.5.

In one approach, when an exemplary embodiment of a decision tree is used, the expression data for a selected set of markers across a training population is standardized to have mean zero and unit variance. The members of the training population are randomly divided into a training set and a test set. For example, in one embodiment, two thirds of the members of the training population are placed in the training set and one third of the members of the training population are placed in the test set. The expression values for a select combination of markers described herein is used to construct the analytical process. Then, the ability for the analytical process to correctly classify members in the test set is determined. In some embodiments, this computation is performed several times for a given combination of markers. In each iteration of the computation, the members of the training population are randomly assigned to the training set and the test set. Then, the quality of the combination of molecular markers is taken as the average of each such iteration of the analytical process computation.

In addition to univariate decision trees in which each split is based on an expression level for a corresponding marker, among the set of markers disclosed herein, or the expression level of two such markers, multivariate decision trees can be implemented as an analytical process. In such multivariate decision trees, some or all of the decisions actually comprise a linear combination of expression levels for a plurality of markers. Such a linear combination can be trained using known techniques such as gradient descent on a classification or by the use of a sum-squared-error criterion. To illustrate such an analytical process, consider the expression: 0.04x₁+0.16x₂<500

Here, x₁ and x₂ refer to two different features for two different markers from among the markers disclosed herein. To poll the analytical process, the values of features x₁ and x₂ are obtained from the measurements obtained from the unclassified subject. These values are then inserted into the equation. If a value of less than 500 is computed, then a first branch in the decision tree is taken. Otherwise, a second branch in the decision tree is taken. Multivariate decision trees are described in Duda, 2001, Pattern Classification, John Wiley & Sons, Inc., New York, pp. 408-409, which is hereby incorporated by reference.

Another approach that can be used in the present invention is multivariate adaptive regression splines (MARS). MARS is an adaptive procedure for regression, and is well suited for the high-dimensional problems addressed by the methods disclosed herein. MARS can be viewed as a generalization of stepwise linear regression or a modification of the CART method to improve the performance of CART in the regression setting. MARS is described in Hastie et al., 2001, The Elements of Statistical Learning, Springer-Verlag, New York, pp. 283-295, which is hereby incorporated by reference in its entirety.

Clustering

In some embodiments, the expression values for a selected set of markers are used to cluster a training set. For example, consider the case in which ten markers are used. Each member m of the training population will have expression values for each of the ten markers. Such values from a member m in the training population define the vector:

X₁ₘX₂ₘX₃ₘX₄ₘX₅ₘX₆ₘX₇ₘX₈ₘX₉ₘX₁₀ₘ

where Xᵢₘ is the expression level of the i^{th} marker in subject m. If there are m organisms in the training set, selection of i markers will define m vectors. Note that the methods disclosed herein do not require that each the expression value of every single marker used in the vectors be represented in every single vector m. In other words, data from a subject in which one of the i^{th} marker is not found can still be used for clustering. In such instances, the missing expression value is assigned either a “zero” or some other normalized value. In some embodiments, prior to clustering, the expression values are normalized to have a mean value of zero and unit variance.

Those members of the training population that exhibit similar expression patterns across the training group will tend to cluster together. A particular combination of markers is considered to be a good classifier in this aspect of the methods disclosed herein when the vectors cluster into the trait groups found in the training population. For instance, if the training population includes healthy patients and atherosclerotic patients, a clustering classifier will cluster the population into two groups, with each group uniquely representing either healthy patients and atherosclerotic patients.

Clustering is described on pages 211-256 of Duda and Hart, Pattern Classification and Scene Analysis, 1973, John Wiley & Sons, Inc., New York, which is hereby incorporated by reference in its entirety for such teachings. As described in Section 6.7 of Duda, the clustering problem is described as one of finding natural groupings in a dataset. To identify natural groupings, two issues are addressed. First, a way to measure similarity (or dissimilarity) between two samples is determined. This metric (similarity measure) is used to ensure that the samples in one cluster are more like one another than they are to samples in other clusters. Second, a mechanism for partitioning the data into clusters using the similarity measure is determined.

Similarity measures are discussed in Section 6.7 of Duda, where it is stated that one way to begin a clustering investigation is to define a distance function and to compute the matrix of distances between all pairs of samples in a dataset. If distance is a good measure of similarity, then the distance between samples in the same cluster will be significantly less than the distance between samples in different clusters. However, as stated on page 215 of Duda, clustering does not require the use of a distance metric. For example, a nonmetric similarity function s(x, x′) can be used to compare two vectors x and x′. Conventionally, s(x, x′) is a symmetric function whose value is large when x and x′ are somehow “similar.” An example of a nonmetric similarity function s(x, x′) is provided on page 216 of Duda.

Once a method for measuring “similarity” or “dissimilarity” between points in a dataset has been selected, clustering requires a criterion function that measures the clustering quality of any partition of the data. Partitions of the data set that extremize the criterion function are used to cluster the data. See page 217 of Duda. Criterion functions are discussed in Section 6.8 of Duda.

More recently, Duda et al., Pattern Classification, 2nd edition, John Wiley & Sons, Inc. New York, has been published. Pages 537-563 describe clustering in detail. More information on clustering techniques can be found in Kaufman and Rousseeuw, 1990, Finding Groups in Data: An Introduction to Cluster Analysis, Wiley, New York, N.Y.; Everitt, 1993, Cluster analysis (3d ed.), Wiley, New York, N.Y.; and Backer, 1995, Computer-Assisted Reasoning in Cluster Analysis, Prentice Hall, Upper Saddle River, N.J. Particular exemplary clustering techniques that can be used with the methods disclosed herein include, but are not limited to, hierarchical clustering (agglomerative clustering using nearest-neighbor algorithm, farthest-neighbor algorithm, the average linkage algorithm, the centroid algorithm, or the sum-of-squares algorithm), k-means clustering, fuzzy k-means clustering algorithm, and Jarvis-Patrick clustering.

### Principal Component Analysis

Principal component analysis (PCA) has been proposed to analyze biomarker data. More generally, PCA can be used to analyze feature value data of markers disclosed herein in order to construct a analytical process that discriminates one class of patients from another (e.g., those who have atherosclerosis and those who do not). Principal component analysis is a classical technique to reduce the dimensionality of a data set by transforming the data to a new set of variable (principal components) that summarize the features of the data. See, for example, Jolliffe, 1986, Principal Component Analysis, Springer, New York, which is hereby incorporated by reference.

A few examples of PCA are as follows. Principal components (PCs) are uncorrelate and are ordered such that the k^{th} PC has the k^{th} largest variance among PCs. The k^{th} PC can be interpreted as the direction that maximizes the variation of the projections of the data points such that it is orthogonal to the first k−1 PCs. The first few PCs capture most of the variation in the data set. In contrast, the last few PCs are often assumed to capture only the residual ‘noise’ in the data.

PCA can also be used to create an analytical process as disclosed herein. In such an approach, vectors for a selected set of markers can be constructed in the same manner described for clustering. In fact, the set of vectors, where each vector represents the expression values for the select markers from a particular member of the training population, can be considered a matrix. In some embodiments, this matrix is represented in a Free-Wilson method of qualitative binary description of monomers (Kubinyi, 1990, 3D QSAR in drug design theory methods and applications, Pergamon Press, Oxford, pp 589-638), and distributed in a maximally compressed space using PCA so that the first principal component (PC) captures the largest amount of variance information possible, the second principal component (PC) captures the second largest amount of all variance information, and so forth until all variance information in the matrix has been accounted for.

Then, each of the vectors (where each vector represents a member of the training population) is plotted. Many different types of plots are possible. In some embodiments, a one-dimensional plot is made. In this one-dimensional plot, the value for the first principal component from each of the members of the training population is plotted. In this form of plot, the expectation is that members of a first group (e.g. healthy patients) will cluster in one range of first principal component values and members of a second group (e.g., patients with atheroclerosis) will cluster in a second range of first principal component values (one of skill in the art would appreciate that the distribution of the marker values need to exhibit no elongation in any of the variables for this to be effective).

In one example, the training population comprises two groups: healthy patients and patients with atherosclerosis. The first principal component is computed using the marker expression values for the selected markers across the entire training population data set. Then, each member of the training set is plotted as a function of the value for the first principal component. In this example, those members of the training population in which the first principal component is positive are the healthy patients and those members of the training population in which the first principal component is negative are atherosclerotic patients.

In some embodiments, the members of the training population are plotted against more than one principal component. For example, in some embodiments, the members of the training population are plotted on a two-dimensional plot in which the first dimension is the first principal component and the second dimension is the second principal component. In such a two-dimensional plot, the expectation is that members of each subgroup represented in the training population will cluster into discrete groups. For example, a first cluster of members in the two-dimensional plot will represent subjects with mild atherosclerosis, a second cluster of members in the two-dimensional plot will represent subjects with moderate atherosclerosis, and so forth.

In some embodiments, the members of the training population are plotted against more than two principal components and a determination is made as to whether the members of the training population are clustering into groups that each uniquely represents a subgroup found in the training population. In some embodiments, principal component analysis is performed by using the R mva package (Anderson, 1973, Cluster Analysis for applications, Academic Press, New York 1973; Gordon, Classification, Second Edition, Chapman and Hall, CRC, 1999.). Principal component analysis is further described in Duda, Pattern Classification, Second Edition, 2001, John Wiley & Sons, Inc.

Nearest Neighbor Classifier Analysis

Nearest neighbor classifiers are memory-based and require no model to be fit. Given a query point x₀, the k training points x₍ᵣ₎, r, . . . , k closest in distance to x₀ are identified and then the point x₀ is classified using the k nearest neighbors. Ties can be broken at random. In some embodiments, Euclidean distance in feature space is used to determine distance as:

d₍ᵢ₎=∥x₍ᵢ₎−x₀∥

Typically, when the nearest neighbor algorithm is used, the expression data used to compute the linear discriminant is standardized to have mean zero and variance 1. For the disclosed methods, the members of the training population are randomly divided into a training set and a test set. For example, in one embodiment, two thirds of the members of the training population are placed in the training set and one third of the members of the training population are placed in the test set. Profiles of a selected set of markers disclosed herein represents the feature space into which members of the test set are plotted. Next, the ability of the training set to correctly characterize the members of the test set is computed. In some embodiments, nearest neighbor computation is performed several times for a given combination of markers. In each iteration of the computation, the members of the training population are randomly assigned to the training set and the test set. Then, the quality of the combination of markers is taken as the average of each such iteration of the nearest neighbor computation.

The nearest neighbor rule can be refined to deal with issues of unequal class priors, differential misclassification costs, and feature selection. Many of these refinements involve some form of weighted voting for the neighbors. For more information on nearest neighbor analysis, see Duda, Pattern Classification, Second Edition, 2001, John Wiley & Sons, Inc; and Hastie, 2001, The Elements of Statistical Learning, Springer, New York, each of which is hereby incorporated by reference in its entirety.

### Evolutionary Methods

Inspired by the process of biological evolution, evolutionary methods of classifier design employ a stochastic search for an analytical process. In broad overview, such methods create several analytical processes—a population—from measurements such as the biomarker generated datasets disclosed herein. Each analytical process varies somewhat from the other. Next, the analytical processes are scored on data across the training datasets. In keeping with the analogy with biological evolution, the resulting (scalar) score is sometimes called the fitness. The analytical processes are ranked according to their score and the best analytical processes are retained (some portion of the total population of analytical processes). Again, in keeping with biological terminology, this is called survival of the fittest. The analytical processes are stochastically altered in the next generation—the children or offspring. Some offspring analytical processes will have higher scores than their parent in the previous generation, some will have lower scores. The overall process is then repeated for the subsequent generation: The analytical processes are scored and the best ones are retained, randomly altered to give yet another generation, and so on. In part, because of the ranking, each generation has, on average, a slightly higher score than the previous one. The process is halted when the single best analytical process in a generation has a score that exceeds a desired criterion value. More information on evolutionary methods is found in, for example, Duda, Pattern Classification, Second Edition, 2001, John Wiley & Sons, Inc.

### Bagging, Boosting, and the Random Subspace Method

Bagging, boosting, the random subspace method, and additive trees are data analysis algorithms known as combining techniques that can be used to improve weak analytical processes. These techniques are designed for, and usually applied to, decision trees, such as the decision trees described above. In addition, such techniques can also be useful in analytical processes developed using other types of data analysis algorithms such as linear discriminant analysis. In addition, Skurichina and Duin provide evidence to suggest that such techniques can also be useful in linear discriminant analysis.

In bagging, one samples the training datasets, generating random independent bootstrap replicates, constructs the analytical processes on each of these, and aggregates them by a simple majority vote in the final analytical process. See, for example, Breiman, 1996, Machine Learning 24, 123-140; and Efron & Tibshirani, An Introduction to Bootstrap, Chapman & Hall, New York, 1993, which is hereby incorporated by reference in its entirety.

In boosting, analytical processes are constructed on weighted versions of the training set, which are dependent on previous analytical process results. Initially, all objects have equal weights, and the first analytical process is constructed on this data set. Then, weights are changed according to the performance of the analytical process. Erroneously classified objects get larger weights, and the next analytical process is boosted on the reweighted training set. In this way, a sequence of training sets and classifiers is obtained, which is then combined by simple majority voting or by weighted majority voting in the final decision. See, for example, Freund & Schapire, “Experiments with a new boosting algorithm,” Proceedings 13th International Conference on Machine Learning, 1996, 148-156.

To illustrate boosting, consider the case where there are two phenotypic groups exhibited by the population under study, phenotype 1 (e.g., poor prognosis patients), and phenotype 2 (e.g., good prognosis patients). Given a vector of molecular markers X, a classifier G(X) produces a prediction taking one of the type values in the two value set: {phenotype 1, phenotype 2}. The error rate on the training sample is

$$err = 1 / N {\sum }_{i = 1}^{N} I ( {y}_{i} \neq G ( {x}_{i} ) )$$

where N is the number of subjects in the training set (the sum total of the subjects that have either phenotype 1 or phenotype 2). For example, if there are 35 healthy patients and 46 sclerotic patients, N is 81.

A weak analytical process is one whose error rate is only slightly better than random guessing. In the boosting algorithm, the weak analytical process is repeatedly applied to modified versions of the data, thereby producing a sequence of weak classifiers Gₘ(x), m=1, 2, . . . , M. The predictions from all of the classifiers in this sequence are then combined through a weighted majority vote to produce the final prediction:

$$G ( x ) = sign ( {\sum }_{m = 1}^{M} {α}_{m} {G}_{m} ( x ) )$$

Here α₁, α₂, . . . , αₘ are computed by the boosting algorithm and their purpose is to weigh the contribution of each respective Gₘ(x). Their effect is to give higher influence to the more accurate classifiers in the sequence.

The data modifications at each boosting step consist of applying weights w₁, w₂, . . . , wₙ to each of the training observations (xᵢ, yᵢ), i=1, 2, . . . , N. Initially all the weights are set to wᵢ=1/N, so that the first step simply trains the analytical process on the data in the usual manner. For each successive iteration m=2, 3, . . . , M the observation weights are individually modified and the analytical process is reapplied to the weighted observations. At stem m, those observations that were misclassified by the analytical process Gₘ₋₁(x) induced at the previous step have their weights increased, whereas the weights are decreased for those that were classified correctly. Thus as iterations proceed, observations that are difficult to correctly classify receive ever-increasing influence. Each successive analytical process is thereby forced to concentrate on those training observations that are missed by previous ones in the sequence.

The exemplary boosting algorithm is summarized as follows:

1. Initialize the observation weights wᵢ=1/N, i=1, 2, . . . , N.

2. For m=1 to M:

(a) Fit an analytical process Gₘ(x) to the training set using weights wᵢ.

(b) Compute

$$err = \frac{{\sum }_{i = 1}^{N} {w}_{i} I ( {y}_{i} \neq {G}_{m} ( {x}_{i} ) )}{{\sum }_{i = 1}^{N} {w}_{i}}$$

(c) Compute αₘ=log((1−errₘ)/errₘ).

(d) Set wᵢ [symbol] wᵢ exp[αₘI(yᵢ≠Gₘ(xᵢ))], i=1, 2, . . . , N.

3. Output

$$G ( x ) = sign \vert {\sum }_{m = i}^{M} {α}_{m} {G}_{m} ( x ) \vert$$

In the algorithm, the current classifier Gₘ(x) is induced on the weighted observations at line 2a. The resulting weighted error rate is computed at line 2b. Line 2c calculates the weight αₘ given to Gₘ(x) in producing the final classifier Gₘ(x) (line 3). The individual weights of each of the observations are updated for the next iteration at line 2d. Observations misclassified by Gₘ(x) have their weights scaled by a factor exp(αₘ), increasing their relative influence for inducing the next classifier Gₘ+I(x) in the sequence. In some embodiments, modifications of the Freund and Schapire, 1997, Journal of Computer and System Sciences 55, pp. 119-139, boosting method are used. See, for example, Hasti et al., The Elements of Statistical Learning, 2001, Springer, New York, Chapter 10. In some embodiments, boosting or adaptive boosting methods are used.

In some embodiments, modifications of Freund and Schapire, 1997, Journal of Computer and System Sciences 55, pp. 119-139, are used. For example, in some embodiments, feature preselection is performed using a technique such as the nonparametric scoring methods of Park et al., 2002, Pac. Symp. Biocomput. 6, 52-63. Feature preselection is a form of dimensionality reduction in which the markers that discriminate between classifications the best are selected for use in the classifier. Then, the LogitBoost procedure introduced by Friedman et al., 2000, Ann Stat 28, 337-407 is used rather than the boosting procedure of Freund and Schapire. In some embodiments, the boosting and other classification methods of Ben-Dor et al., 2000, Journal of Computational Biology 7, 559-583 are used in the disclosed methods. In some embodiments, the boosting and other classification methods of Freund and Schapire, 1997, Journal of Computer and System Sciences 55, 119-139, are used.

In the random subspace method, classifiers are constructed in random subspaces of the data feature space. These classifiers are usually combined by simple majority voting in the final decision rule (i.e., analytical process). See, for example, Ho, “The Random subspace method for constructing decision forests,” IEEE Trans Pattern Analysis and Machine Intelligence, 1998; 20(8): 832-844.

### Other Statistical Analysis Algorithms

As indicated at the beginning of this section, the statistical techniques described above are merely examples of the types of algorithms and models that can be used to identify a preferred group of markers to include in a dataset and to generate an analytical process that can be used to generate a result using the dataset. Further, combinations of the techniques described above and elsewhere can be used either for the same task or each for a different task. Some combinations, such as the use of the combination of decision trees and boosting, have been described. However, many other combinations are possible. By way of example, other statistical techniques in the art such as Projection Pursuit and Weighted Voting can be used to identify a preferred group of markers to include in a dataset and to generate an analytical process that can be used to generate a result using the dataset.

### Determining Optimum Number of Dataset Components to be Evaluated in Analytical Process

When using the learning algorithms described above to develop a predictive model, one of skill in the art may select a subset of markers, i.e. at least 3, at least 4, at least 5, at least 6, up to the complete set of markers, to define the analytical process. Usually a subset of markers will be chosen that provides for the needs of the quantitative sample analysis, e.g. availability of reagents, convenience of quantitation, etc., while maintaining a highly accurate predictive model.

The selection of a number of informative markers for building classification models requires the definition of a performance metric and a user-defined threshold for producing a model with useful predictive ability based on this metric. For example, the performance metric may be the AUC, the sensitivity and/or specificity of the prediction as well as the overall accuracy of the prediction model.

The predictive ability of a model may be evaluated according to its ability to provide a quality metric, e.g. AUC or accuracy, of a particular value, or range of values. In some embodiments, a desired quality threshold is a predictive model that will classify a sample with an accuracy of at least about 0.7, at least about 0.75, at least about 0.8, at least about 0.85, at least about 0.9, at least about 0.95, or higher. As an alternative measure, a desired quality threshold may refer to a predictive model that will classify a sample with an AUC (area under the curve) of at least about 0.7, at least about 0.75, at least about 0.8, at least about 0.85, at least about 0.9, or higher.

As is known in the art, the relative sensitivity and specificity of a predictive model can be “tuned” to favor either the selectivity metric or the sensitivity metric, where the two metrics have an inverse relationship. The limits in a model as described above can be adjusted to provide a selected sensitivity or specificity level, depending on the particular requirements of the test being performed. One or both of sensitivity and specificity may be at least about at least about 0.7, at least about 0.75, at least about 0.8, at least about 0.85, at least about 0.9, or higher.

As described in Examples 5, 11 and 12, various methods are used in a training model. The selection of a subset of markers may be via a forward selection or a backward selection of a marker subset. The number of markers to be selected is that which will optimize the performance of a model without the use of all the markers. One way to define the optimum number of terms is to choose the number of terms that produce a model with desired predictive ability (e.g. an AUC>0.75, or equivalent measures of sensitivity/specificity) that lies no more than one standard error from the maximum value obtained for this metric using any combination and number of terms used for the given algorithm.

### Use of Results Generated by Analytic Process

As described above, datasets from containing quantitative data for components of the dataset are inputted into an analytic process and used to generate a result. The result can be any type of information useful for making an atherosclerotic classification, e.g. a classification, a continuous variable, or a vector. For example, the value of a continuous variable or vector may be used to determine the likelihood that a sample is associated with a particular classification.

Atherosclerotic classification refer to any type of information or the generation of any type of information associated with an atherosclerotic condition, for example, diagnosis, staging, assessing extent of atherosclerotic progression, prognosis, monitoring, therapeutic response to treatments, screening to identify compounds that act via similar mechanisms as known atherosclerotic treatments, prediction of pseudo-coronary calcium score, stable (i.e., angina) vs. unstable (i.e., myocardial infarction), identifying complications of atherosclerotic disease, etc.

Further details regarding the appropriate type of reference or training data to be used to develop predictive models for various atherosclerotic classifications and how to use such models to predict certain types of atherosclerotic classifications is described below.

In a preferred embodiment, the result is used for diagnosis or detection of the occurrence of an atherosclerosis, particularly where such atherosclerosis is indicative of a propensity for myocardial infarction, heart failure, etc. In this embodiment, a reference or training set containing “healthy” and “atherosclerotic” samples is used to develop a predictive model. A dataset, preferably containing protein expression levels of markers indicative of the atherosclerosis, is then inputted into the predictive model in order to generate a result. The result may classify the sample as either “healthy” or “atherosclerotic”. In other embodiments, the result is a continuous variable providing information useful for classifying the sample, e.g., where a high value indicates a high probability of being an “atherosclerotic” sample and a low value indicates a low probability of being a “healthy” sample.

In other embodiments, the result is used for atherosclerosis staging. In this embodiment, a reference or training dataset containing samples from individuals with disease at different stages is used to develop a predictive model. The model may be a simple comparison of an individual dataset against one or more datasets obtained from disease samples of known stage or a more complex multivariate classification model. In certain embodiments, inputting a dataset into the model will generate a result classifying the sample from which the dataset is generated as being at a specified cardiovascular disease stage. Similar methods may be used to provide atherosclerosis prognosis, except that the reference or training set will include data obtained from individuals who develop disease and those who fail to develop disease at a later time.

In other embodiments, the result is used determine response to atherosclerotic disease treatments. In this embodiment, the reference or training dataset and the predictive model is the same as that used to diagnose atherosclerosis (samples of from individuals with disease and those without). However, the instead of inputting a dataset composed of samples from individuals with an unknown diagnosis, the dataset is composed of individuals with known disease which have been administered a particular treatment and it is determined whether the samples trend toward or lie within a normal, healthy classification versus an atherosclerotic disease classification.

In another embodiment, the result is used for drug screening, i.e., identifying compounds that act via similar mechanisms as known atherosclerotic drug treatments (Examples 6-7). In this embodiment, a reference or training set containing individuals treated with a known atherosclerotic drug treatment and those not treated with the particular treatment can be used develop a predictive model. A dataset from individuals treated with a compound with an unknown mechanism is input into the model. If the result indicates that the sample can be classified as coming from a subject dosed with a known atherosclerotic drug treatment, then the new compound is likely to act via the same mechanism.

In preferred embodiments, the result is used to determine a “pseudo-coronary calcium score,” which is a quantitative measure that correlates to coronary calcium score (CCS). CCS is a clinical cardiovascular disease screening technique which measures overall atherosclerotic plaque burden. Various different types of imaging techniques can be used to quantitate the calcium area and density of atherosclerotic plaques. When electron-beam CT and multidetector CT are used, CCS is a function of the x-ray attenuation coefficient and the area of calcium deposits. Typically, a score of 0 is considered to indicate no atherosclerotic plaque burden. >0 to 10 to indicate minimal evidence of plaque burden, 11 to 100 to indicate at least mild evidence of plaque burden, 101 to 400 to indicate at least moderate evidence of plaque burden, and over 400 as being extensive evidence of plaque burden. CCS used in conjunction with traditional risk factors improves predictive ability for complications of cardiovascular disease. In addition, the CCS is also capable of acting an independent predictor of cardiovascular disease complications. Budoff et al., “Assessment of Coronary Artery Disease by Cardiac Computed Tomography,” Circulation 113: 1761-1791 (2006).

A reference or training set containing individuals with high and low coronary calcium scores can be used develop a model, e.g., Example 8, for predicting the pseudo-coronary calcium score of an individual. This predicted pseudo-coronary calcium score is useful for diagnosing and monitoring atherosclerosis. In some embodiments, the pseudo-coronary calcium score is used in conjunction with other known cardiovascular diagnosis and monitoring methods, such as actual coronary calcium score derived from imaging techniques to diagnose and monitor cardiovascular disease.

One of skill will also recognize that the results generated using these methods can be used in conjunction with any number of the various other methods known to those of skill in the art for diagnosing and monitoring cardiovascular disease.

### Reagents and Kits

Also provided are reagents and kits thereof for practicing one or more of the above-described methods. The subject reagents and kits thereof may vary greatly. Reagents of interest include reagents specifically designed for use in production of the above described expression profiles of circulating protein markers associated with atherosclerotic conditions.

One type of such reagent is an array or kit of antibodies that bind to a marker set of interest. A variety of different array formats are known in the art, with a wide variety of different probe structures, substrate compositions and attachment technologies. Representative array or kit compositions of interest include or consist of reagents for quantitation of at least two, at least three, at least four, at least five or more protein markers are selected from M-CSF, eotaxin, IP-10, MCP-1, MCP-2, MCP-3, MCP-4, IL-3, IL-5, IL-7, IL-8, MIP1a, TNFa, and RANTES.

In other embodiments, a representative array or kit includes or consists of reagents for quantitation of at least three protein markers selected from the following group: f MCP-1, MCP-2, MCP-3, MCP-4, eotaxin, IP-10, M-CSF, IL-3, TNFa, Ang-2, IL-5, IL-7, and IGF-1. The at least three protein markers may comprise or consist of a marker set selected from the following group: MCP-1, IGF-1, TNFa; MCP-1, IGF-1, M-CSF; ANG-2, IGF-1, M-CSF; and MCP-4, IGF-1, M-CSF.

In other embodiments, a representative array or kit includes or consists of reagents for quantitation of at least four protein markers selected from the following group: MCP-1, MCP-2, MCP-3, MCP-4, eotaxin, IP-10, M-CSF, IL-3, TNFa, Ang-2, IL-5, IL-7, and IGF-1. The at least four protein markers comprise or consist of MCP-1, MCP-2, MCP-3, MCP-4, eotaxin, IP-10, M-CSF, IL-3, TNFa, Ang-2, IL-5, IL-7, and IGF-1; MCP-1, IGF-1, TNFa, IL-5; MCP-1, IGF-1, M-CSF, MCP-2; ANG-2, IGF-1, M-CSF, IL-5; MCP-1, IGF-1, TNFa, MCP-2; and MCP-4, IGF-1, M-CSF, IL-5.

In other embodiments, a representative array or kit includes or consists of reagents for quantitation of at least five protein markers selected from the following group: MCP-1, MCP-2, MCP-3, MCP-4, eotaxin, IP-10, M-CSF, IL-3, TNFa, Ang-2, IL-5, IL-7, and IGF-1. The at least five markers may comprise or consist of a marker set selected from the following group: MCP-1, MCP-2, MCP-3, MCP-4, eotaxin, IP-10, M-CSF, IL-3, TNFa, Ang-2, IL-5, IL-7, and IGF-1; MCP-1, IGF-1, TNFa, IL-5, M-CSF; MCP-1, IGF-1, M-CSF, MCP-2, IP-10; ANG-2, IGF-1, M-CSF, IL-5, TNFa; MCP-1, IGF-1, TNFa, MCP-2, IP-10; MCP-4, IGF-1, M-CSF, IL-5, TNFa; and MCP-4, IGF-1, M-CSF, IL-5, MCP-2.

The kits may further include a software package for statistical analysis of one or more phenotypes, and may include a reference database for calculating the probability of classification. The kit may include reagents employed in the various methods, such as devices for withdrawing and handling blood samples, second stage antibodies, ELISA reagents; tubes, spin columns, and the like.

In addition to the above components, the subject kits will further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address which may be used via the internet to access the information at a removed site. Any convenient means may be present in the kits.

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

### Example 1 Classification of “Healthy” vs. “Disease” using TIMP1 and RANTES Markers

To investigate the multimarker approach in distinguishing subjects with active coronary artery disease from those without disease, we utilized a large clinical epidemiological study which included 400 cases of clinically significant ASCVD and 930 control subjects. The study was designed to examine risk factors and other novel determinants of atherosclerosis. Serum samples collected at the time of enrollment were used for simultaneous measurement of multiple inflammatory markers using a protein microarray. The exact methodology used for the pilot studies was utilized here (discussed in details in examples in WO97/002677 “Methods and Compositions for Diagnosis and Monitoring of Atherosclerotic Cardiovascular Disease”). Concentrations of a subset of the analytes tested were significantly higher in case subjects. Classification algorithms using the serum expression profile of these markers accurately stratified CAD subjects compared to controls. Moreover, the unique signature pattern of the biomarkers significantly improved the predictive capacity of other known markers of CAD. This larger trial replicated our prior findings but also provided with more examples for use of multimarker approach for accurate prediction and diagnosis of atherosclerotic cardiovascular disease and its various clinical sequelae.

The selection of a number of informative markers for building classification models requires the definition of a performance metric and a user-defined threshold for producing a model with useful predictive ability based on this metric. In the following section we defined the target quantity to be the “area under the curve” (AUC), the sensitivity and/or specificity of the prediction as well as the overall accuracy of the prediction model. This is the approach we used for selecting the number of terms for building a predictive model in the absence of any clinical variables and/or adjusting factors. The process was as follows: We first randomly split our training data into ten groups, each group containing subjects identified as “Healthy” or “Diseased” in proportion to the number of these labels in the complete sample. Each subject was represented by its 26 marker measurements and the label that identifies the state of disease (absent, i.e. “Healthy” of present, i.e. “Diseased”). We chose nine of the groups and for each of the 26 markers (TIMP1, RANTES, MCP-1, IGF-1, TNFa, IL-5, M-CSF, MCP-2, IP10, MCP-4, IL3, IFNg, Ang-2, IL-7, IL-10, Eotaxin, IL-2, IL-4, ICAM-1, IL-6, IL-12p40, MIP1a, IL-5, MCP-3, IL13, IL1b) we trained a model using a given supervised algorithm, e.g., Linear Discriminant Analysis, Quadratic Discriminant Analysis, Logistic Regression on all the data of the 9 groups (i.e. we created a training supergroup). We then applied the model to the tenth group that was excluded from the training procedure and we estimated the testing error “e” and or a number of prediction quality measures described earlier. We repeated the same process 10 times, sampling randomly 9 groups each time for generating a training sample and using the 10^{th} group for estimating the testing error “e” and the prediction quality measures. From the sample of the 10 numbers we then estimated the expected value for each of the prediction quality measures and/or error, as a well the variance of our estimates. Given these values, the marker that improves the average prediction ability of the model as chosen as the first term in the model.

As an alternative, we also used another measure of improvement instead of the average value of the prediction quality measure, for example we instead selected the term with the highest value of the ratio of the expected quality measure to its variance estimate. Once the first term was added to the model, we repeated the process for the remaining markers that did not make it in the current selection step. Thus, in the second step we repeated the aforementioned calculations for the remaining markers. The selection of the second model term was accomplished by choosing the term that mostly improves our target prediction quality measure or using some combination of the expected value of the current model minus the new model normalized by the errors of those measures.

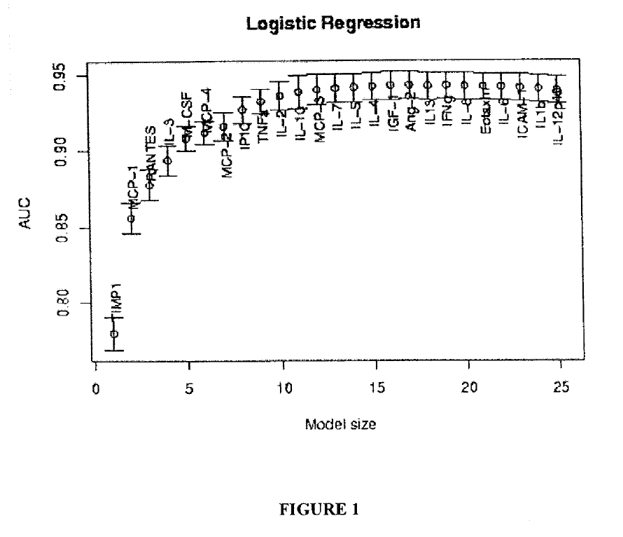

FIG. 1 shows the results of applying this process to a set of 1300 subjects. We selected the threshold of AUC>0.85 as our target prediction quality measure and we selected the terms using a Logistic Regression model. The quality threshold was satisfied using the following marker: TIMP1, MCP-1 and RANTES.

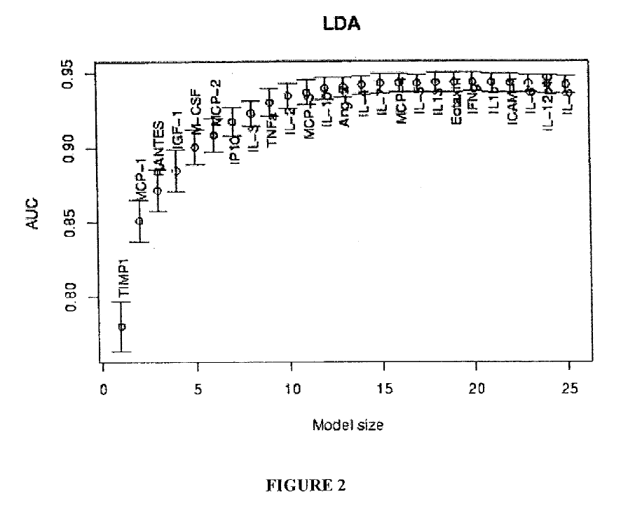

FIG. 2 shows the results of selecting the terms using a Linear Discriminant Analysis model while keeping the discovery sample and quality thresholds the same. The comparison with the previous example indicates that the two models agree on the selected terms that satisfy our performance criteria.

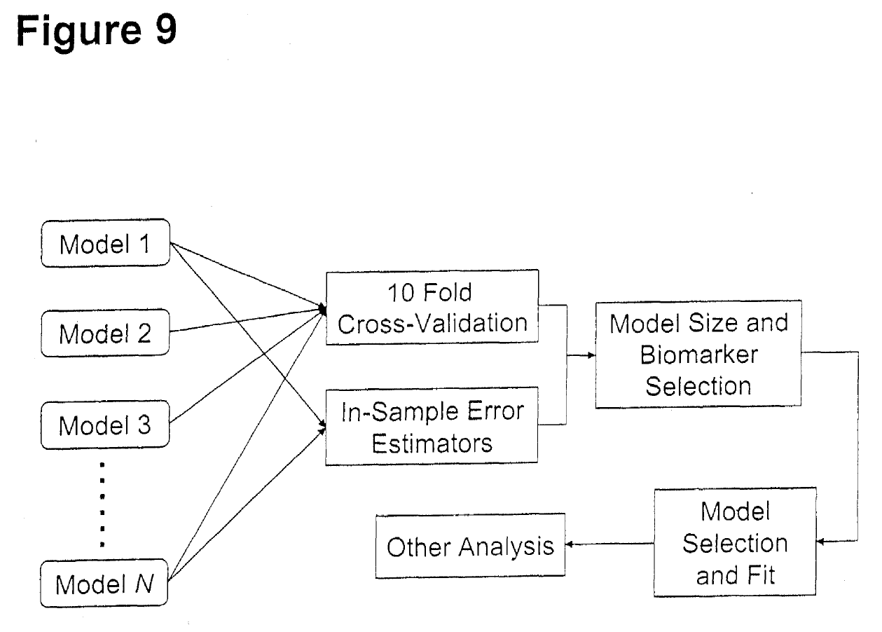

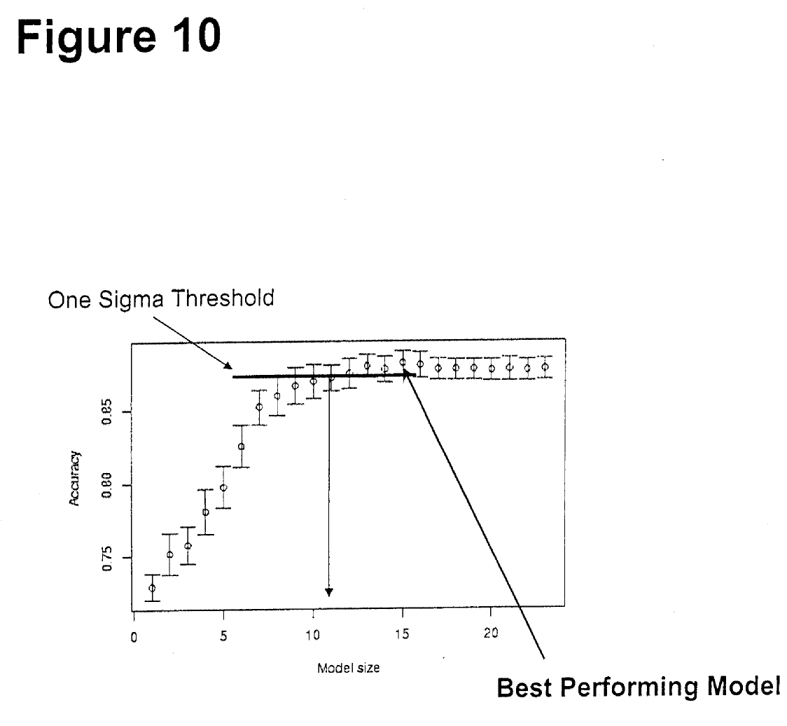

Another option for term addition, in a forward fashion, to each model is to use the misclassification error, accuracy or log-likelihood of the data. The process was started by adding the first term in the model. This term was selected so that (i) the misclassification rate was the smallest from all the rates obtained with any single marker, (ii) the accuracy was the highest or (iii) the log-likelihood of the data was the highest. Using 10-fold cross-validation the expected value of this metric and its standard error was estimated. Once the model with the first term was created, we again selected the next term by: a) creating a two term model where the best term from the previous step was combined with each one of the remaining available markers and b) by finding the marker that in combination with the term that was already in the model provided the smallest misclassification error among the remaining markers, the highest accuracy or the highest increase in log-likelihood. The expected out-of sample expected value and its standard error for the model of size two were again estimated using a 10-fold cross-validation. We continuously added terms until we have used all the terms and estimate the expected value and standard error for all nested models. Then we chose the smallest model that was within one standard error from the best value of the quality measure used for the term selection. The overall approach is summarized in FIG. 9. In this figure, Model 1,2, . . . N represents any of the classification algorithms described earlier. The 10-fold cross validation can be any of 3-fold, 5-fold, 10-fold, . . . (N−1)-fold (leave-one-out) cross-validation. A demonstration of this approach using accuracy as the quality criterion is shown in FIG. 10.

### Example 2

### Classification of Patients with Coronary Calcium Score Above and Below Given Clinically Relevant Thresholds

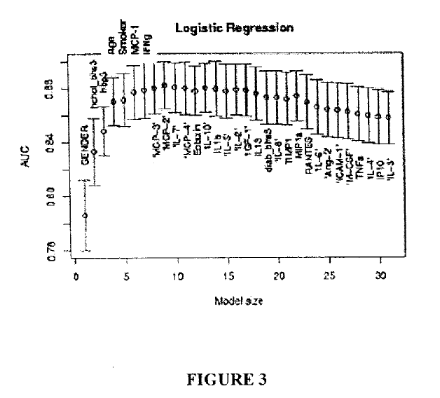

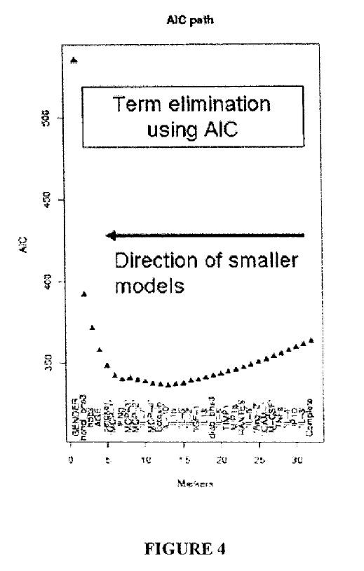

Based on the literature, subjects with CCS<10 are in low risk for adverse events while subjects with CCS>400 are at high risk for adverse events. Based on these criteria we built classification models for these two populations to predict high and low pseudo-coronary calcium score. We assigned the label “upper” for the subjects with CCS>400 and the label “lower” for the subjects with CCS<10. We then used the AIC criterion to identify the terms of the Logistic Regression model that best separates the two groups. For this application, we allowed clinical variables to be included in the model if selected based on the AIC criterion. FIG. 3 shows the order in which terms were dropped. The clinical variables are the most significant predictors but the minimum of the selection path is obtained only when protein markers are included (MCP-1, IFNg.). FIG. 4 shows the selection process for the same classification problem using the cross-validation approach.

### Additional Examples

The following Examples demonstrate various applications using twenty four of the markers from Example 1 (excluding RANTES and TIMP1). Any of the following Examples can be performed using RANTES and/or TIMP1 as additional biomarkers.

### Example 3

### AIC Selection Criteria

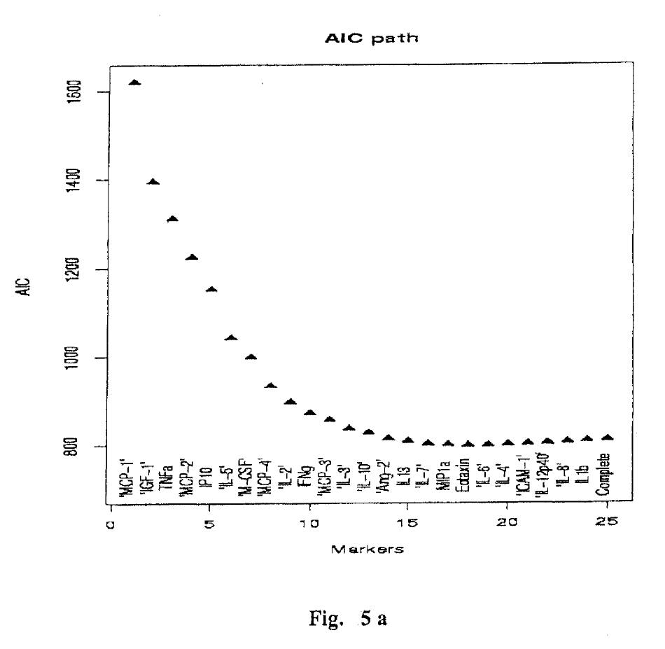

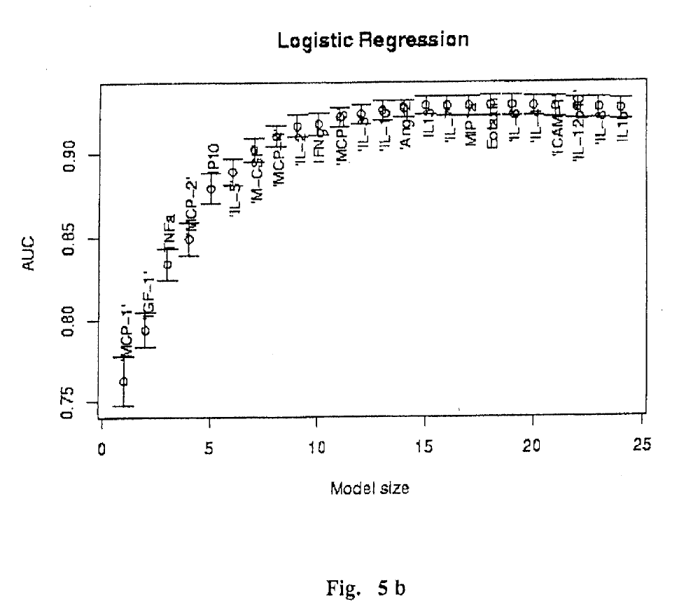

As an example of a different selection criterion, we present the results obtained using the AIC criterion within the framework of a Logistic Regression model. This criterion is usually used in the context of selecting the optimum number of terms for a Logistic Regression model. The criterion balances the error increase due to the removal of a term with the reduction of the number of degrees of freedom that this term contributed to the model. Usually, the process of term elimination starts with the full model and terminates when the removal of a term increases the AIC value. The results of term elimination as a function of the AIC criterion are presented in FIG. 5a (the term elimination process is presented past the optimum point). The AUC predictions for a model incorporating increasing number of terms are presented in FIG. 5b. The addition of terms in the aforementioned model is performed in the reverse order of term removal from the complete model, i.e., a model including only 24 of the above markers that the application of the AIC criterion dictates in the term selection process. The latter approach produces a Logistic Regression model with expected AUC>0.75 using at least one marker (MCP-1).

The process of term selection can be accomplished either with a forward selection (first, second and third examples within this working example) or a backward selection (fourth example within this working example), or a forward/backward selection strategy. This strategy allows for testing of all the terms that have been removed in a previous step in the current reduced model.

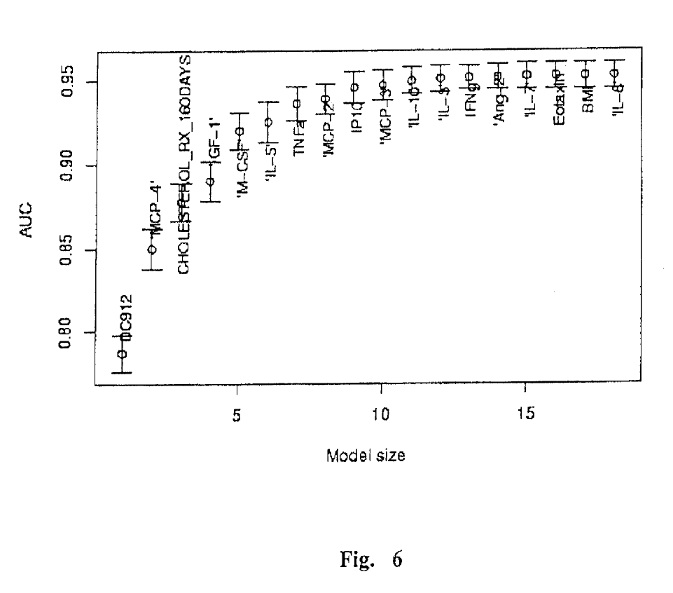

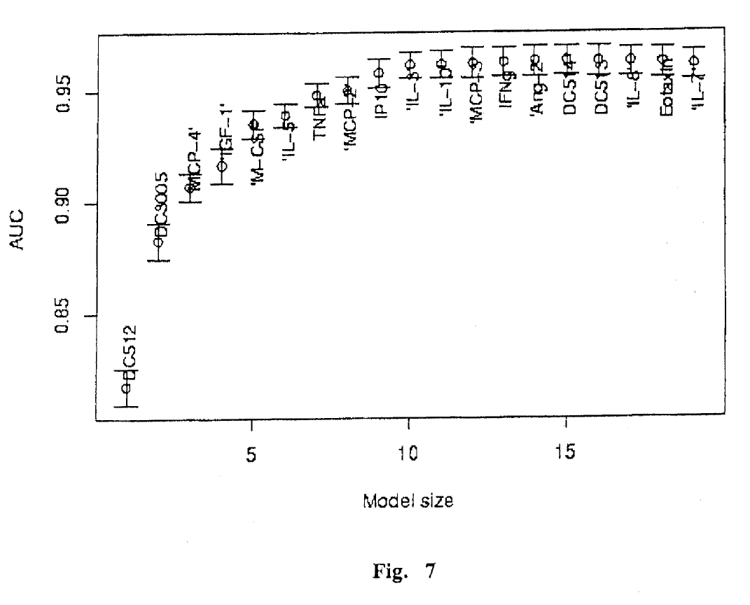

The same selection process can be extended to include both markers and clinical variables. The next two figures, present the results for the case that the candidate variables for a Logistic Regression model include “Hyperlipidemia” (DC912) and “Use of lipid-lowering medication within 160 days before index day” (FIG. 6) or “Statin use,” “ACE blockers use” (FIG. 7) along with all 16 markers. These examples demonstrate that the markers in the set of at least 3 markers required for obtaining an AUC>0.75 can be replaced with clinical variables in the set. The combination of Hyperlipidemia (DC912) and MCP-4 produces a model with expected value of AUC˜0.85.

Using the aforementioned methods we can also select the number of markers that will optimize the performance of a model without the use of all the markers. One way to define the optimum number of terms is to choose the number of terms that produce a model with average predictive ability (measured as AUC, or equivalent measures of sensitivity/specificity) that lies no more than one standard error from the maximum value obtained for any combination and number of terms used for the given algorithm. Looking back at FIG. 7, a Logistic Regression model that includes the following markers satisfies these requirements: Beta Blockers (“DC512”), Statins (“DC3005”), MCP-4, IGF-1, M-CSF, IL-5, MCP-2, IP-10.

### Example 4

### ACE Inhibitor Response Prediction Models

Using the methods described in Examples 1 and 3, we derived models using Logistic Regression or Linear Discriminant Analysis that classify samples according to the use of ACE inhibitors. These models were adjusted for the status of the subject (Control or Case) since the overall level of the markers depends on whether we deal with a healthy individual or not. The models find use in a variety of methods such as, e.g., screening compounds to identify other agents that act as ACE inhibitors or on convergent pathways, and for monitoring the efficacy of ACE inhibitor therapy. In the first example, the compound is provided to a mammalian subject, one or more samples are taken from the subject and datasets are obtained from the sample(s). The datasets are run through an ACE Inhibitor Response Prediction model and the results are used to classify the sample. If the sample is classified as coming from a subject dosed with an ACE inhibitor, then the compound is likely to be a presumptive ACE inhibitor. In the second example, one or more samples are obtained from a subject and datasets from those samples are run through an ACE Inhibitor Response Prediction model. If the sample is classified as coming from a subject dosed with an ACE inhibitor then the therapy is likely to be efficacious. If multiple samplings over time indicate time dependent changes in the value of a predictor obtained from the model, then the therapeutic efficacy of the medication therapy is likely changing, the direction of the change being indicated by a predictor value trending more toward the medication use classification or the no-medication use classification. The protein markers used in the exemplified models are set out in Tables 2 and 3, below, along with the models' performance characteristics.

TABLE 2
ACE Inhibitor Prediction Model 1.

| Logistic Regression | | | | | |
|---|---|---|---|---|---|
| Variables used: | mis-classification | AUC | sensitivity | specificity | accuracy |
| MCP-1, IGF-1, TNFa, MCP-2, IP10, IL-5, M- | 0.365 | 0.688 | 0.641 | 0.632 | 0.635 |
| CSF, MCP-4, MCP-3, IL-3, Ang-2, IL- | | | | | |
| 7, Eotaxin | | | | | |

TABLE 3
ACE Inhibitor Prediction Model 2.

| Linear Discriminant Analysis | | | | | |
|---|---|---|---|---|---|
| Variables used: | mis-classification | AUC | sensitivity | specificity | accuracy |
| MCP-1, IGF-1, TNFa, MCP-2, IP10, IL-5, M- | 0.376 | 0.689 | 0.632 | 0.620 | 0.624 |
| CSF, MCP-4, MCP-3, IL-3, Ang-2, IL- | | | | | |
| 7, Eotaxin | | | | | |

### Example 5

### ACE Inhibitor or Statin Use Prediction Models

Using the methods described in Examples 1 and 3, we derived models using Logistic Regression or Linear Discriminant Analysis that classify samples according to the use of ACE inhibitors or statins. These models were adjusted for the status of the subject (Control or Case) since the overall level of the markers depends on whether we deal with a healthy individual or not. The models find use in a variety of methods such as, e.g., screening compounds to identify other agents that act as ACE inhibitors or statins or on convergent pathways, and for monitoring the efficacy of ACE inhibitor or statin therapy. In the first example, the compound is provided to a mammalian subject, one or more samples are taken from the subject and datasets are obtained from the sample(s). The datasets are run through an ACE Inhibitor or Statin Use Prediction model and the results are used to classify the sample. If the sample is classified as coming from a subject dosed with an ACE inhibitor or statin, then the compound is likely to be a presumptive ACE inhibitor or statin. In the second example, one or more samples are obtained from a subject and datasets from those samples are run through an ACE Inhibitor or Statin Use Prediction model. If the sample is classified as coming from a subject dosed with an ACE inhibitor or statin then the therapy is likely to be efficacious. If multiple samplings over time indicate time dependent changes in the value of a predictor obtained from the model, then the therapeutic efficacy of the medication therapy is likely changing, the direction of the change being indicated by a predictor value trending more toward the medication use classification or the no-medication use classification. The protein markers used in the exemplified models are set out in Tables 4 and 5, below, along with the models' performance characteristics.

### Biomarker Profile for Medication Use Responsiveness

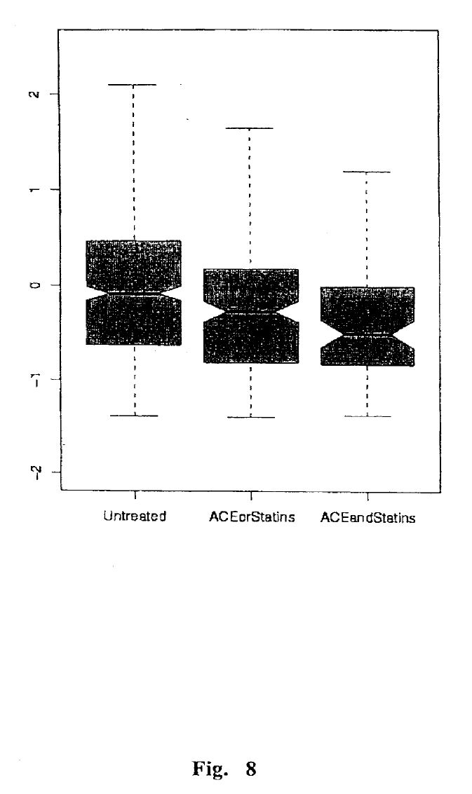

We demonstrate that a panel of markers can be used for monitoring the medication effect on the level of inflammation of a subject. Inspecting the distribution of values for a number of markers (IL-2,IL-5,IL-4) we demonstrate a dosage effect as a function of the number of medications that a control subject is treated with (i.e. no medication vs. one medication vs. two medications). As an example for this approach, we use three medication responsive markers as a panel (IL-2,IL-4 and IL-5). In order to create a single combined score, we create a linear discriminant analysis model where the response variable takes the following levels: “Untreated”, “ACE or Statin”, “ACE and Statin” and we use the first discriminant variate as a surrogate for a combined score. FIG. 8 presents the results from the subjects that are considered “Healthy” (“Controls”) as boxplots for each of the three “treatment” groups. The grey sections of each boxplot extend from the first to the third quantile of the value distribution for each class. The “notches:” around the medians are included for facilitating visual inspection of differences in the level of the median between the classes. The whiskers extend to 1.5 times the interquantile distance. The outliers have not been included in the graph. Clearly the combined score shows a downward trend with increased number of medications. The fact that the notches for the groups are barely overlapping indicates that the differences in the median are rather significant. A panel of biomarkers performs better than any single biomarker alone.

A similar analysis can be performed by creating a single score from multiple markers using Hottelling's T² method. In this case we can estimate the covariance matrix from the data for the untreated group and calculate the “distance” of each subject based on Hottelling's formula. The later approach can be used not only for creating a “combined distance” from many markers for monitoring medication dosage effect but also for hypothesis testing of the dosage effect. (see Hotelling, H. (1947). Multivariate Quality Control. In C. Eisenhart, M. W. Hastay, and W. A. Wallis, eds. Techniques of Statistical Analysis. New York: McGraw-Hill., herein incorporated by reference).

TABLE 4
ACE Inhibitor or Statin Prediction Model 1.

| Logistic Regression | | | | | |
|---|---|---|---|---|---|
| Variables used: | mis-classification | AUC | sensitivity | specificity | accuracy |
| MCP-1, IGF-1, TNFa, MCP-2, IP10, IL-5, | 0.318 | 0.751 | 0.643 | 0.723 | 0.682 |
| M-CSF, MCP-4, MCP-3, IL-3, Ang-2, IL- | | | | | |
| 7, Eotaxin | | | | | |

TABLE 5
ACE Inhibitor or Statin Prediction Model 2.

| Linear Discriminant Analysis | | | | | |
|---|---|---|---|---|---|
| Variables used: | mis-classification | AUC | sensitivity | specificity | accuracy |
| MCP-1, IGF-1, TNFa, MCP-2, IP10, IL-5, M- | 0.320 | 0.754 | 0.686 | 0.673 | 0.680 |
| CSF, MCP-4, MCP-3, IL-3, Ang-2, IL- | | | | | |
| 7, Eotaxin | | | | | |

### Example 6

### Coronary Calcium Score Prediction Models

Using the methods described in Examples 1 and 3, we derived models using Logistic Regression or Linear Discriminant Analysis that classify samples according to a predicted coronary calcium score. The protein markers used in the exemplified models are set out in Tables 6 and 7, below, along with the models' performance characteristics.

TABLE 6
Coronary Calcium Score Prediction Model 1.

| Logistic Regression | | | | | |
|---|---|---|---|---|---|
| Variables used: | mis-classification | AUCc | sensitivity | specificity | accuracy |
| MCP-1, IGF-1, TNFa, MCP-2, IP10, IL-5, M- | 0.470 | 0.536 | 0.567 | 0.500 | 0.530 |
| CSF, MCP-4, MCP-3, IL-3, Ang-2, IL- | | | | | |
| 7, Eotaxin | | | | | |

TABLE 7
Coronary Calcium Score Prediction Model 2.

| Linear Discriminant Analysis | | | | | |
|---|---|---|---|---|---|
| Variables used: | mis-classification | AUC | sensitivity | specificity | accuracy |
| MCP-1, IGF-1, TNFa, MCP-2, IP10, IL-5, M- | 0.461 | 0.560 | 0.578 | 0.505 | 0.539 |
| CSF, MCP-4, MCP-3, IL-3, Ang-2, IL- | | | | | |
| 7, Eotaxin | | | | | |

### Example 7

### Stable vs. Unstable Atherosclerotic Disease Prediction Models

Using the methods described in Examples 1 and 3, we derived models using Logistic Regression or Linear Discriminant Analysis that classify samples into stable (i.e., angina) or unstable (i.e., myocardial infarction) categories. The protein markers used in the exemplified models are set out in Tables 8 and 9, below, along with the models' performance characteristics.

TABLE 8
Stable vs. Unstable Disease Prediction Model 1.

| Logistic Regression | | | | | |
|---|---|---|---|---|---|
| Variables used: | mis-classification | AUC | sensitivity | specificity | accuracy |
| MCP-1, IGF-1, TNFa, MCP-2, IP10, IL-5, M- | 0.438 | 0.566 | 0.563 | 0.562 | 0.562 |
| CSF, MCP-4, MCP-3, IL-3, Ang-2, IL- | | | | | |
| 7, Eotaxin | | | | | |

TABLE 9
Stable vs. Unstable Disease Prediction Model 2.

| Linear Discriminant Analysis | | | | | |
|---|---|---|---|---|---|
| Variables used: | mean cv error | AUC | sensitivity | specificity | accuracy |
| MCP-1, IGF-1, TNFa, MCP-2, IP10, IL-5, M- | 0.444 | 0.577 | 0.583 | 0.529 | 0.556 |
| CSF, MCP-4, MCP-3, IL-3, Ang-2, IL- | | | | | |
| 7, Eotaxin | | | | | |

### Example 8

### Disease vs. Healthy Control Prediction Models

Using the methods described in Examples 1 and 3, we derived models using Logistic Regression or Linear Discriminant Analysis that classify samples into disease (i.e., angina or myocardial infarction) or healthy control categories. The protein markers used in the exemplified models are set out in Tables 10 and 11, below, along with the models' performance characteristics. Tables 10 and 11 also indicate how the performance of the models change as combinations of markers are substituted.

TABLE 10
Disease vs. Control Prediction Model 1.

| Linear Discriminant Analysis | | | | | |
|---|---|---|---|---|---|
| Variables used: | mis-classification | AUC | sensitivity | specificity | accuracy |
| MCP-1, IGF-1, TNFa, MCP-2, IP10, IL-5, M- | 0.158 | 0.915 | 0.847 | 0.840 | 0.842 |
| CSF, MCP-4, MCP-3, IL-3, Ang-2, IL- | | | | | |
| 7, Eotaxin | | | | | |
| MCP-1, IGF-1, TNFa | 0.245 | 0.827 | 0.804 | 0.733 | 0.755 |
| MCP-1, IGF-1, M-CSF | 0.235 | 0.825 | 0.786 | 0.756 | 0.765 |
| Ang-2, IGF-1, M-CSF | 0.258 | 0.798 | 0.718 | 0.753 | 0.742 |
| MCP-4, IGF-1, M-CSF | 0.258 | 0.789 | 0.721 | 0.750 | 0.742 |
| MCP-1, IGF-1, TNFa, IL-5 | 0.225 | 0.850 | 0.817 | 0.757 | 0.775 |
| MCP-1, IGF-1, M-CSF, MCP-2 | 0.227 | 0.842 | 0.801 | 0.760 | 0.773 |
| Ang-2, IGF-1, M-CSF, IL-5 | 0.239 | 0.816 | 0.754 | 0.764 | 0.761 |
| MCP-1, IGF-1, TNFa, MCP-2 | 0.240 | 0.842 | 0.792 | 0.746 | 0.760 |
| MCP-1, IGF-1, TNFa, IL-5, M-CSF | 0.213 | 0.867 | 0.837 | 0.765 | 0.787 |
| MCP-1, IGF-1, IP10, MCP-2, M-CSF | 0.184 | 0.874 | 0.807 | 0.821 | 0.816 |
| Ang-2, IGF-1, TNFa, IL-5, M-CSF | 0.216 | 0.855 | 0.807 | 0.774 | 0.784 |
| MCP-1, IGF-1, TNFa, MCP-2, IP10 | 0.203 | 0.878 | 0.784 | 0.802 | 0.797 |
| MCP-4, IGF-1, M-CSF, TNFa, IL-5 | 0.221 | 0.855 | 0.812 | 0.765 | 0.779 |
| MCP-4, IGF-1, M-CSF, MCP-2, IL-5 | 0.246 | 0.807 | 0.736 | 0.761 | 0.754 |

TABLE 11
Disease vs. Control Prediction Model 2.

| Logistic Regression | | | | | |
|---|---|---|---|---|---|
| Variables used: | mis-classification | AUC | sensitivity | specificity | accuracy |
| MCP-1, IGF-1, TNFa, MCP-2, IP10, IL-5, M- | 0.153 | 0.916 | 0.859 | 0.841 | 0.847 |
| CSF, MCP-4, MCP-3, IL-3, Ang-2, IL- | | | | | |
| 7, Eotaxin | | | | | |
| MCP-1, IGF-1, TNFa | 0.237 | 0.835 | 0.804 | 0.745 | 0.763 |
| MCP-1, IGF-1, M-CSF | 0.239 | 0.831 | 0.789 | 0.749 | 0.761 |
| Ang-2, IGF-1, M-CSF | 0.257 | 0.799 | 0.734 | 0.747 | 0.743 |
| MCP-4, IGF-1, M-CSF | 0.258 | 0.792 | 0.733 | 0.745 | 0.742 |
| MCP-1, IGF-1, TNFa, IL-5 | 0.221 | 0.856 | 0.826 | 0.759 | 0.779 |
| MCP-1, IGF-1, M-CSF, MCP-2 | 0.236 | 0.845 | 0.794 | 0.750 | 0.764 |
| Ang-2, IGF-1, M-CSF, IL-5 | 0.243 | 0.813 | 0.766 | 0.754 | 0.757 |
| MCP-1, IGF-1, TNFa, MCP-2 | 0.235 | 0.849 | 0.784 | 0.757 | 0.765 |
| MCP-1, IGF-1, TNFa, IL-5, M-CSF | 0.212 | 0.868 | 0.832 | 0.769 | 0.788 |
| MCP-1, IGF-1, IP10, MCP-2, M-CSF | 0.187 | 0.876 | 0.804 | 0.816 | 0.813 |
| Ang-2, IGF-1, TNFa, IL-5, M-CSF | 0.220 | 0.855 | 0.801 | 0.771 | 0.780 |
| MCP-1, IGF-1, TNFa, MCP-2, IP10 | 0.202 | 0.881 | 0.794 | 0.799 | 0.798 |
| MCP-4, IGF-1, M-CSF, TNFa, IL-5 | 0.223 | 0.857 | 0.807 | 0.764 | 0.777 |
| MCP-4, IGF-1, M-CSF, MCP-2, IL-5 | 0.258 | 0.810 | 0.734 | 0.746 | 0.742 |

### Example 9

### Classification using an LDA Model

We classified a patient into a “Control” or “Disease” category based on the values of the following markers MCP-1, IGF-1 and TNFa. The costs of misclassification are taken to be equal for the two classes. Based on an LDA approach, a new subject with values x of the aforementioned markers is categorized into the “Disease” category if the left side of equation (1) is greater than the right side of the equation where:

a) index 2 corresponds to the “Disease” state

b) index 1 corresponds to the “Control” state

c) N is the total size of the training set

d) N1,N2 are the number of “Control” and “Disease” subjects in the training set

e) Σ is the covariance matrix as estimated from the training set

f) μ_{1,2} are the mean vectors of the “Control” and “Disease” sample respectively

$${x}^{T} {\hat{\sum }}^{- 1} ( \text{?} ) > \frac{1}{2} {\overline{μ}}_{2}^{T} {\hat{\sum }}^{- 1} {\overline{u}}_{2} - \frac{1}{2} {\overline{μ}}_{1}^{T} {\hat{\sum }}^{- 1} {\overline{μ}}_{1} + log ( {N}_{1} / N ) log ( {N}_{2} / N ) \\ \text{?} \text{indicates text missing or illegible when filed} \qquad ( 1 )$$

In order to build an LDA model for the prediction we used a training set containing the three marker values for 398 subjects that were identified as “Control” and 398 subjects that were identified as “Disease.” The marker values are first log 10 transformed and the resulting values are used to estimate the required terms of Eq. 1. The covariance matrix and mean marker vectors for the training set are equal to:

Covariance matrix:

| | MCP-1 | IGF-1 | TNFa |
|---|---|---|---|
| MCP-1 | 0.124155 | 0.069587 | 0.06659 |
| IGF-1 | 0.069587 | 1.321971 | 0.664374 |
| TNFa | 0.06659 | 0.664374 | 0.565535 |

Mean marker vectors for “Control” and “Disease” states:

| | | | |
|---|---|---|---|
| Control | 1.891552 | 2.830981 | 0.781913 |
| Disease | 1.223976 | 2.324683 | 0.990313 |

The inverse of the covariance matrix that is needed in equation 1 is:

| | V1 | V2 | V3 |
|---|---|---|---|
| 1 | 8.607599 | 0.13735 | −1.17487 |
| 2 | 0.13735 | 1.848967 | −2.18828 |
| 3 | −1.17487 | −2.18828 | 4.477304 |

We classified a subject with the following values (transformed using a log 10transformation):

Subject 1:

| MCP-1 | IGF-1 | TNFa |
|---|---|---|
| 0.716998 | 1.316101 | 0.287882 |

Based on these values and Eq. 1, the left side of the equation is equal to: 0.5291794 while the right side of the equation is equal to 3.232524. Based on the fact that the left side is less than the right side, the subject was classified into the “Control” category.

We classified a second subject with the following log 10transformed marker values:

Subject 2:

| MCP-1 | IGF-1 | TNFa |
|---|---|---|
| 1.991509 | 1.1113031 | 0.536339 |

Based on these values and using equation 1, the left side is equal to 4.461167 and the right hand side remains 3.232524. Based on this comparison the subject was classified into the “Disease” category.

Reference for this and the following example is made to “The elements of Statistical Learning. Data Mining, Inference and Prediction”, Hastie, T., Tibshirani, R., Friedman, J., Springer Series in Statistics, 2001), herein incorporated by reference.

### Example 10

### Classification using a Logistic Regression Model

We classified a patient into a “Control” or “Disease” category based on the values of the following markers MCP-1, IGF-1 and M-CSF. The costs of misclassification are taken to be equal for the two classes. Based on a Logistic Regression approach, a new subject with values x of the aforementioned markers will be categorized as Disease if the log ratio of the posterior probabilities of class k (=Disease) to class K(=Control) is greater than zero, otherwise it is categorized as Control (Equation 2).

$$log \frac{Pr ( G = k \vert X = x )}{Pr ( G = K \vert X = x )} = {β}_{k 0} + {β}_{k}^{T} x . \qquad ( 2 )$$

In order to fit a Logistic Regression model we used a training set composed of 398 subjects identified as “Control” and 398 subjects identified as “Disease.” The values of the three markers for each subject were first log 10transformed. The Logistic Regression fit provides the following coefficients:

| b0 | b1 | b2 | b3 |
|---|---|---|---|
| −4.95059 | 3.334 | −1.27675 | 1.279328 |

A new subject with the following values for the three markers was classified:

| | MCP-1 | IGF-1 | M-CSF |
|---|---|---|---|
| Subject 1 | 1.679931 | 3.493781 | 1.169145 |

The following calculation b0+b1*‘MCP-1’+b2*‘IGF-1’+b3*‘M-CSF’ equals −2.031. Based on the previous discussion this subject has a linear predictor value less than zero and was classified into the “Control” category.

Another subject was classified, based on the following values:

| | MCP-1 | IGF-1 | M-CSF |
|---|---|---|---|
| Subject 2 | 2.108252 | 1.7149 | 0.539566 |

Using the same coefficients and formula the linear predictor equals 0.5799186 and Subject 2 was classified into the “Disease” category.

Each publication cited in this specification is hereby incorporated by reference in its entirety for all purposes. In addition to those publications listed throughout the body of this specification, the following also is hereby incorporated by reference in its entirety for all purposes: Tabibiazar R, Wagner R A, Deng A, Tsao P S, Quertermous T. Proteomic profiles of serum inflammatory markers accurately predict atherosclerosis in mice. Physiol Genomics. 2006 Apr. 13; 25(2):194-202.

1. A method for generating a result useful in diagnosing and monitoring atherosclerotic disease using a sample obtained from a mammalian subject, comprising:
obtaining a dataset associated with said sample, wherein said dataset comprises protein expression levels for at least three markers selected from the group consisting of the proteins RANTES, TIMP1, MCP-1, MCP-2, MCP-3, MCP-4, eotaxin, IP-10, M-CSF, IL-3, TNFa, Ang-2, IL-5, IL-7, IGF-1, sVCAM, sICAM-1, E-selectin, P-selection, interleukin-6, interleukin-18, creatine kinase, LDL, oxLDL, LDL particle size, Lipoprotein(a), troponin I, troponin T, LPPLA2, CRP, HDL, Triglyceride, insulin, BNP, fractalkine, osteopontin, osteoprotegerin, oncostatin-M, Myeloperoxidase, ADMA, PAI-1 (plasminogen activator inhibitor), SAA (circulating amyloid A), t-PA (tissue-type plasminogen activator), sCD40 ligand, fibrinogen, homocysteine, D-dimer, leukocyte count, heart-type fatty acid binding protein, Lipoprotein (a), MMP1, Plasminogen, folate, vitamin B6, Leptin, soluble thrombomodulin, PAPPA, MMP9, MMP2, VEGF, PIGF, HGF, vWF, and cystatin C, wherein one of the at least three protein markers is RANTES or TIMP1; and
inputting said dataset into an analytical process that uses said data to generate a result useful in diagnosing and monitoring atherosclerotic disease.

2. A method for generating a result useful in diagnosing and monitoring atherosclerotic disease using a sample obtained from a mammalian subject, comprising:
obtaining a dataset associated with said sample, wherein said dataset comprises protein expression levels for at least three protein markers selected from the group consisting of RANTES, TIMP1, MCP-1, MCP-2, MCP-3, MCP-4, eotaxin, IP-10, M-CSF, IL-3, TNFa, Ang-2, IL-5, IL-7, and IGF-1, wherein one of the at least three protein markers is RANTES or TIMP1; and
inputting said dataset into an analytical process that uses said data to generate a result useful in diagnosing and monitoring atherosclerotic disease.

3. The method of claim 1 wherein said result is a classification, a continuous variable or a vector.

4. The method of claim 3 wherein the classification comprises two or more classes.

5. The method of claim 4 wherein the classification is a pseudo coronary calcium score and the two or more classes are a low coronary calcium score and a high coronary calcium score.

6. The method of claim 1 wherein said analytical process is a linear algorithm, a quadratic algorithm, a polynomial algorithm, a decision tree algorithm, a voting algorithm, a Linear Discriminant Analysis model, a support vector machine classification algorithm, a recursive feature elimination model, a prediction analysis of microarray model, a Logistic Regression model, a CART algorithm, a FlexTree algorithm, a LART algorithm, a random forest algorithm, a MART algorithm, or Machine Learning algorithms.

7. The method of claim 1, wherein said analytical process comprises use of a predictive model.

8. The method of claim 1, wherein said analytical process comprises comparing said obtained dataset with a reference dataset.

9. The method of claim 8, wherein said reference dataset comprises protein expression levels obtained from one or more healthy control subjects, or comprises protein expression levels obtained from one or more subjects diagnosed with an atherosclerotic disease.

10. The method of claim 8, further comprising obtaining a statistical measure of a similarity of said obtained dataset to said reference dataset.

11. The method of claim 8, wherein said statistical measure is derived from a comparison of at least three parameters of said obtained dataset to corresponding parameters from said reference dataset.

12. A method for classifying a sample obtained from a mammalian subject, comprising:
obtaining a dataset associated with said sample, wherein said dataset comprises protein expression levels for at least three protein markers selected from the group consisting of RANTES, TIMP1, MCP-1, MCP-2, MCP-3, MCP-4, eotaxin, IP-10, M-CSF, IL-3, TNFa, Ang-2, IL-5, IL-7, and IGF-1, wherein one of the at least three protein markers is RANTES or TIMP1;
inputting said dataset into an analytical process that uses said data to classify said sample, wherein said classification is selected from the group consisting of an atherosclerotic cardiovascular disease classification, a healthy classification, a medication exposure classification, a no medication exposure classification, a low coronary calcium score and a high coronary calcium score; and
classifying said sample according to the output of said process.

13. The method of claim 1, wherein said analytical process comprises use of a predictive model.

14. The method of claim 1, wherein said analytical process comprises comparing said obtained dataset with a reference dataset.

15. The method of claim 14, wherein said reference dataset comprises protein expression levels obtained from one or more healthy control subjects, or comprises protein expression levels obtained from one or more subjects diagnosed with an atherosclerotic disease.

16. The method of claim 14, further comprising obtaining a statistical measure of a similarity of said obtained dataset to said reference dataset.

17. The method of claim 16, wherein said statistical measure is derived from a comparison of at least three parameters of said obtained dataset to corresponding parameters from said reference dataset.

18. The method of claim 1, wherein said at least three protein markers comprise a marker set selected from the group consisting of RANTES, TIMP1, MCP-1, IGF-1, TNFa, M-CSF, Ang-2, and MCP-4.

19. The method of claim 1, wherein said dataset comprises protein expression levels for at least four protein markers selected from the group consisting of RANTES, TIMP1, MCP-1, MCP-2, MCP-3, MCP-4, eotaxin, IP-10, M-CSF, IL-3, TNFa, Ang-2, IL-5, IL-7, and IGF-1.

20. The method of claim 19, wherein said at least four protein markers comprise a marker set selected from the group consisting of RANTES, TIMP1, MCP-1, IGF-1, TNFa, IL-5; MCP-1, IGF-1, M-CSF, MCP-2; ANG-2, IGF-1, M-CSF, IL-5; MCP-1, IGF-1, TNFa, MCP-2; and MCP-4, IGF-1, M-CSF, IL-5.

21. The method of claim 1, wherein said dataset comprises protein expression levels for at least five markers selected from the group consisting of RANTES, TIMP1, MCP-1, MCP-2, MCP-3, MCP-4, eotaxin, IP-10, M-CSF, IL-3, TNFa, Ang-2, IL-5, IL-7, and IGF-1.

22. The method of claim 21, wherein said at least five protein markers are selected from the group consisting of RANTES, TIMP1, MCP-1, IGF-1, TNFa, IL-5, M-CSF; MCP-1, IGF-1, M-CSF, MCP-2, IP-10; ANG-2, IGF-1, M-CSF, IL-5, TNFa; MCP-1, IGF-1, TNFa, MCP-2, IP-10; MCP-4, IGF-1, M-CSF, IL-5, TNFa; and MCP-4, IGF-1, M-CSF, IL-5, MCP-2.

23. A method for classifying a sample obtained from a mammalian subject, comprising:
obtaining a dataset associated with said sample, wherein said dataset comprises protein expression levels for at least three protein markers selected from the group consisting of MCP1, MCP2, MCP3, MCP4, Eotaxin, IP10, MCSF, IL3, TNFα, ANG2, IL5, IL7, IGF1, IL10, INFγ, VEGF, MIP1a, RANTES, IL6, IL8, ICAM-1, TIMP1, CCL19, TCA4/6kine/CCL21, CSF3, TRANCE, IL2, IL4, IL13, Il1b, CXCL1/GRO1, GROalpha, IL12, and Leptin, wherein one of the at least three protein markers is RANTES or TIMP1;
inputting said data into a predictive model that uses said data to classify said sample, wherein said classification is selected from the group consisting of an atherosclerotic cardiovascular disease classification, a healthy classification, a medication exposure classification, a no medication exposure classification, wherein said predictive model has at least one quality metric of at least 0.7 for classification; and
classifying said sample according to the output of said predictive model.

24. The method of claim 23, wherein said predictive model has a quality metric of at least 0.8 for classification.

25. The method of claim 24, wherein said predictive model has a quality metric of at least 0.9 for classification.

26. The method of claim 23, wherein said quality metric is selected from AUC and accuracy.

27. The method of claim 23, wherein the limits of said predictive model are adjusted to provide at least one of sensitivity or specificity of at least 0.7.

28. The method of claim 25, wherein the limits of said predictive model are adjusted to provide at least one of sensitivity or specificity of at least 0.7.

29. The method of claim 1, wherein said atherosclerotic cardiovascular disease classification is selected from the group consisting of coronary artery disease, myocardial infarction, and angina.

30. The method of claim 1, further comprising using said classification for atherosclerosis diagnosis, atherosclerosis staging, atherosclerosis prognosis, vascular inflammation levels, assessing extent of atherosclerosis progression, monitoring a therapeutic response, predicting a coronary calcium score, or distinguishing stable from unstable manifestations of atherosclerotic disease.

31. The method of claim 1, wherein said dataset further comprises quantitative data for one or more clinical indicia.

32. The method of claim 31, wherein said one or more clinical indicia are selected from the group consisting of age, gender, LDL concentration, HDL concentration, triglyceride concentration, blood pressure, body mass index, CRP concentration, coronary calcium score, waist circumference, tobacco smoking status, previous history of cardiovascular disease, family history of cardiovascular disease, heart rate, fasting insulin concentration, fasting glucose concentration, diabetes status, and use of high blood pressure medication.

33. The method of claim 1, wherein said sample comprises blood or a blood derivative.

34. The method of claim 1, wherein said analytic process comprises using a Linear Discriminant Analysis model, a support vector machine classification algorithm, a recursive feature elimination model, a prediction analysis of microarray model, a Logistic Regression model, a CART algorithm, a FlexTree algorithm, a LART algorithm, a random forest algorithm, a MART algorithm, or Machine Learning algorithms.

35. The method of claim 34, wherein said process comprises using a Linear Discriminant Analysis model or a Logistic Regression model, and said model comprises terms selected to provide a quality metric greater than 0.75.

36. The method of claim 1, further comprising obtaining a plurality of classifications for a plurality of samples obtained at a plurality of different times from said subject.
